(19) United States
(12) Patent Application Publication
(10) Pub. No.: US 2025/0048921 A1
(43) Pub. Date: Feb. 6, 2025

(54) COMPOUND FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si, Gyeonggi-do (KR)

(72) Inventors: Wook KIM, Suwon-si, Gyeonggi-do (KR); Yoonman LEE, Suwon-si, Gyeonggi-do (KR); Seonyeong GWAK, Suwon-si, Gyeonggi-do (KR); Suheon IM, Suwon-si, Gyeonggi-do (KR); Mijin LEE, Suwon-si, Gyeonggi-do (KR); II Geun HONG, Suwon-si, Gyeonggi-do (KR); Hanill LEE, Suwon-si, Gyeonggi-do (KR)

(21) Appl. No.: 18/717,179

(22) PCT Filed: Dec. 26, 2022
(86) PCT No.: PCT/KR2022/021295

§ 371 Date: Jun. 6, 2024

(30) Foreign Application Priority Data
Dec. 30, 2021 (KR) 10-2021-0193107

(51) Int. Cl. H10K 85/60; C09K 11/06

(52) U.S. Cl. CPC H10K 85/654; C09K 11/06; H10K 85/622; H10K 85/6572; H10K 85/6574

(57) ABSTRACT

Disclosed are a compound for an organic optoelectronic device represented by Chemical Formula 1, an organic optoelectronic device including the same, and a display device.

The contents of Chemical Formula 1 are as defined in the detailed description.

### TECHNICAL FIELD

A compound for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a compound for an organic optoelectronic device capable of implementing an organic optoelectronic device having low driving, high efficiency, and a long life-span.

Another embodiment provides an organic optoelectronic device including the compound.

Another embodiment provides a display device including the organic optoelectronic device.

### Technical Solution

According to an embodiment, a compound for an organic optoelectronic device represented by Chemical Formula 1 is provided.

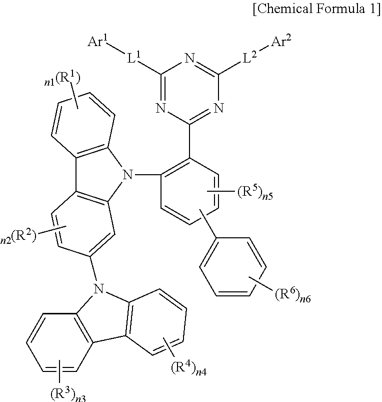

In Chemical Formula 1,
Ar¹ and Ar² are each independently a substituted or unsubstituted C6 to C30 aryl group,
L¹ and L² are each independently a single bond or a substituted or unsubstituted C6 to C30 arylene group,
R¹ to R⁴ are each independently hydrogen, deuterium, or a substituted or unsubstituted C6 to C30 aryl group,
R⁵ and R⁶ are each independently hydrogen or deuterium,
n¹, n³, and n⁴ are each independently one of integers of 1 to 4,
n2 and n5 are one of the integers from 1 to 3, and
n6 is one of integers of 1 to 5.

According to another embodiment, an organic optoelectronic device includes an anode and a cathode facing each other, and at least one organic layer between the anode and the cathode, wherein the organic layer includes a light emitting layer and the light emitting layer includes the aforementioned compound for an organic optoelectronic device.

According to another embodiment, a display device including the organic optoelectronic device is provided.

### Advantageous Effects

An organic optoelectronic device having low driving, high efficiency, and a long life-span may be realized.

### DESCRIPTION OF THE DRAWINGS

FIG. 1 is a cross-sectional view illustrating an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### MODE FOR INVENTION

Hereinafter, embodiments of the present invention are described in detail. However, these embodiments are exemplary, the present invention is not limited thereto and the present invention is defined by the scope of claims.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present invention, the “substituted” refers to replacement of at least one hydrogen of a substituent or a compound by deuterium, a C1 to C20 alkyl group, a C6 to C30 aryl group, or a cyano group. In specific example of the present invention, the “substituted” refers to replacement of at least one hydrogen of a substituent or a compound by deuterium, a C1 to C5 alkyl group, a C6 to C18 aryl group, or a cyano group. In specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quaterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted napbthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

A compound for an organic optoelectronic device according to an embodiment is represented by Chemical Formula 1.

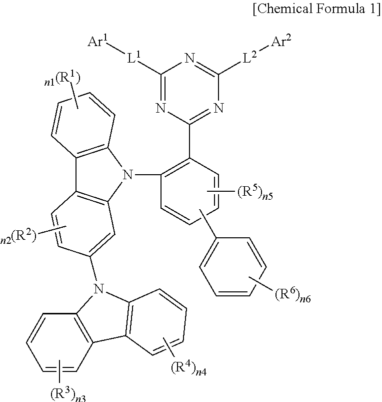

In Chemical Formula 1,

Ar¹ and Ar² are each independently a substituted or unsubstituted C6 to C30 aryl group,

L¹ and L² are each independently a single bond or a substituted or unsubstituted C6 to C30 arylene group,

R¹ to R⁴ are each independently hydrogen, deuterium, or a substituted or unsubstituted C6 to C30 aryl group,

R⁵ and R⁶ are each independently hydrogen or deuterium,

n¹, n³, and n⁴ are each independently one of integers of 1 to 4,

n2 and n5 are one of the integers from 1 to 3, and

n6 is one of integers of 1 to 5.

The compound represented by Chemical Formula 1 has a structure including triazine and ortho-carbazole-substituted deuterium-substituted or unsubstituted biphenyl, and has particularly excellent energy transfer efficiency to phosphorescent dopant, and therefore it can be used as an advantageous material as a phosphorescent host.

In addition, as carbazole is additionally substituted for the ortho-carbazole, an dihedral angle increases due to steric hindrance between the triazine and the carbazole-substituted ortho-carbazole, and the two moieties are twisted. As a result, the HOMO energy level and the LUMO energy level are mostly separated without overlap, and fast energy transfer is possible using a small ΔEst, showing high efficiency characteristics, especially when applied as a phosphorescent host.

In addition, the life-span can be improved by reducing a side reaction path in the excited state, and the presence of ortho-isomers lowers the deposition temperature by about 10% compared to para or meta, thereby minimizing degradation and decomposition.

When n¹ is 2 or more, each R¹ may be the same or different from each other.

When n2 is 2 or more, each R² may be the same or different from each other.

When n³ is 2 or more, each R³ may be the same or different from each other.

When n⁴ is 2 or more, each R⁴ may be the same or different from each other.

When n5 is 2 or more, each R⁵ may be the same or different from each other.

When n6 is 2 or more, each R⁶ may be the same or different from each other.

As an example, Chemical Formula 1 may be represented by Chemical Formula 1-1.

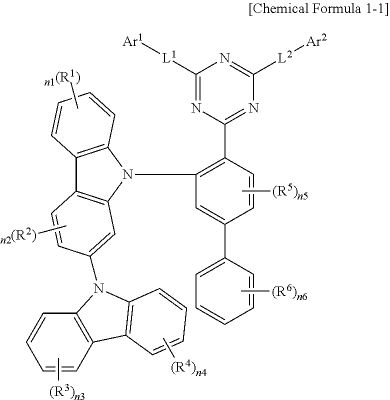

In Chemical Formula 1-1,
the definitions of Ar¹, Ar², R¹ to R⁶, L¹, L², and n1 to n6 are the same as described above.

Para-biphenyl structure included in Chemical Formula 1-1 can accelerate electron mobility compared to mono-phenyl and induce stability of electrons through resonance effect, thereby improving life-span, especially when applied as a phosphorescent host.

In addition, by substituting triazine and biscarbazole at the ortho position of the para-biphenyl, the dihedral angle increases due to steric hindrance between triazine and biscarbazole, and the triazine moiety and the biscarbazole moiety twist each other.

This means that the HOMO energy level and the LUMO energy level are largely separated without overlap, and fast energy transfer is possible using a small ΔEst, showing high efficiency characteristics, especially when applied as a phosphorescent host.

In addition, the side reaction path in the excited state is reduced, which increases the life-span, especially when applied as a phosphorescent host.

For example, R¹ to R⁴ may each independently be hydrogen, deuterium, a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, or a substituted or unsubstituted triphenylene group.

As a specific example, R¹ to R⁴ may each independently be hydrogen, deuterium, a substituted or unsubstituted phenyl group, a substituted or unsubstituted biphenyl group, or a substituted or unsubstituted naphthyl group.

For example, R¹ to R⁴ may each independently be hydrogen, deuterium, or an unsubstituted phenyl group.

For example, Ar¹ and Ar² may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted dibenzofuranyl group, or a substituted or unsubstituted dibenzothiophenyl group.

As a specific example, for example, *-L¹-Ar¹ and *-L²-Ar² may each independently be selected from the substituents listed in Group I.

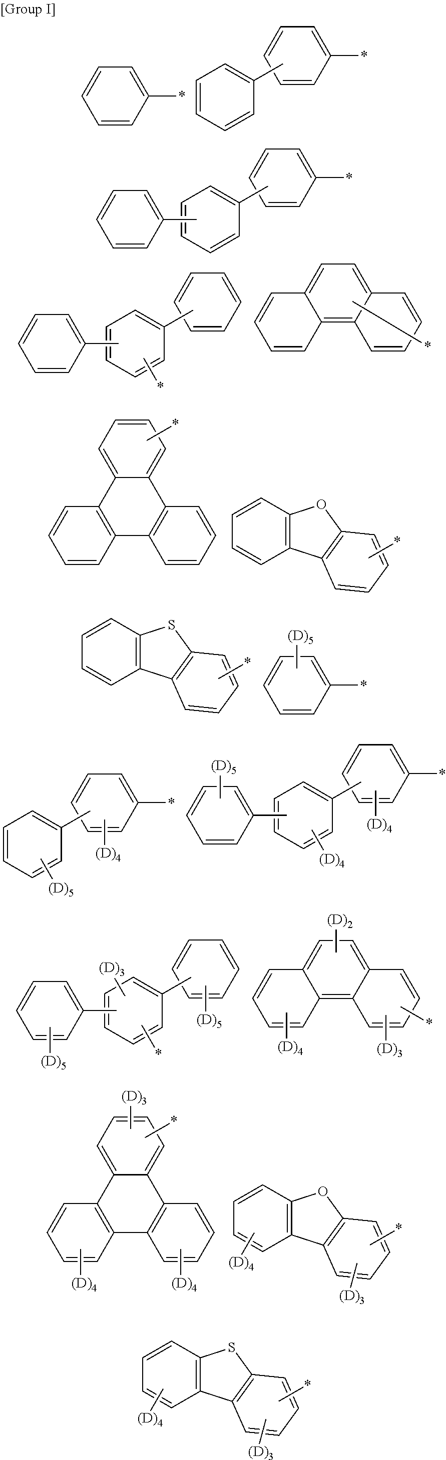

In Group I, * is a linking point, and
the substituents listed in Group I may be unsubstituted or substituted with an additional substituent.

The additional substituent may be deuterium, a cyano group, a C1 to C10 alkyl group, or a C6 to C12 aryl group.

In one example, the additional substituent may be deuterium, a C1 to C5 alkyl group, a phenyl group, a biphenyl group, or a naphthyl group.

For example, Ar¹ and Ar² may each independently be a substituted or unsubstituted phenyl group, a substituted or unsubstituted biphenyl group, or a substituted or unsubstituted naphthyl group.

In the most specific embodiment, the compound for an organic optoelectronic device represented by Chemical Formula 1 may be one selected from the compounds listed in Group 1, but is not limited thereto.

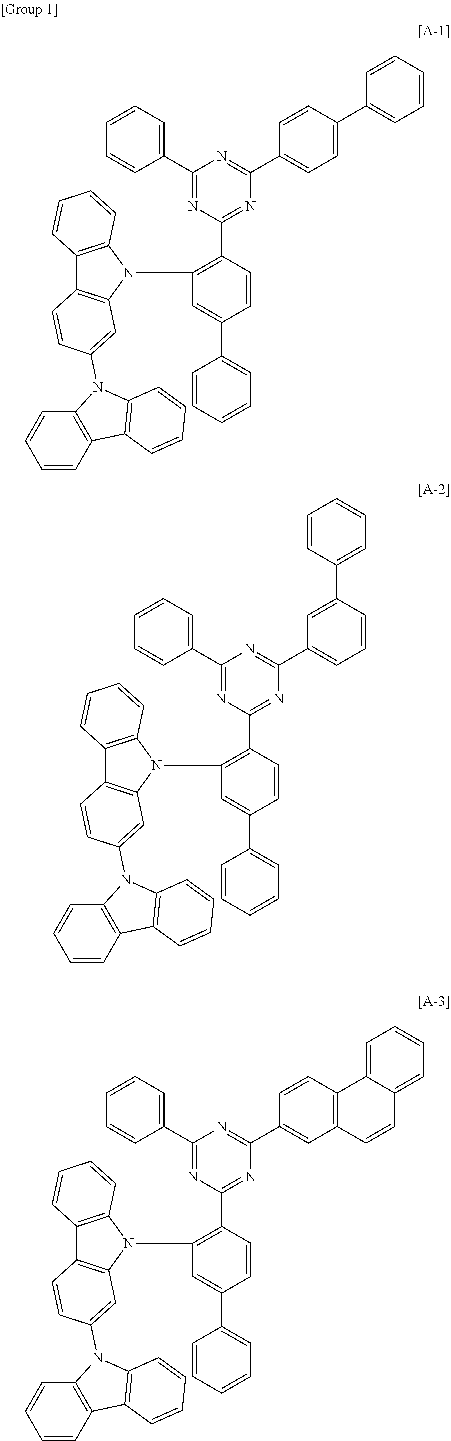 [SMILES: c1ccc(-c2ccc(-c3nc(-c4ccccc4)nc(-c4ccc(-c5ccccc5)cc4-n4c5ccccc5c5ccc(-n6c7ccccc7c7ccccc76)cc54)n3)cc2)cc1 ; c1ccc(-c2cccc(-c3nc(-c4ccccc4)nc(-c4ccc(-c5ccccc5)cc4-n4c5ccccc5c5ccc(-n6c7ccccc7c7ccccc76)cc54)n3)c2)cc1 ; c1ccc(-c2ccc(-c3nc(-c4ccccc4)nc(-c4ccc5c(ccc6ccccc65)c4)n3)c(-n3c4ccccc4c4ccc(-n5c6ccccc6c6ccccc65)cc43)c2)cc1]

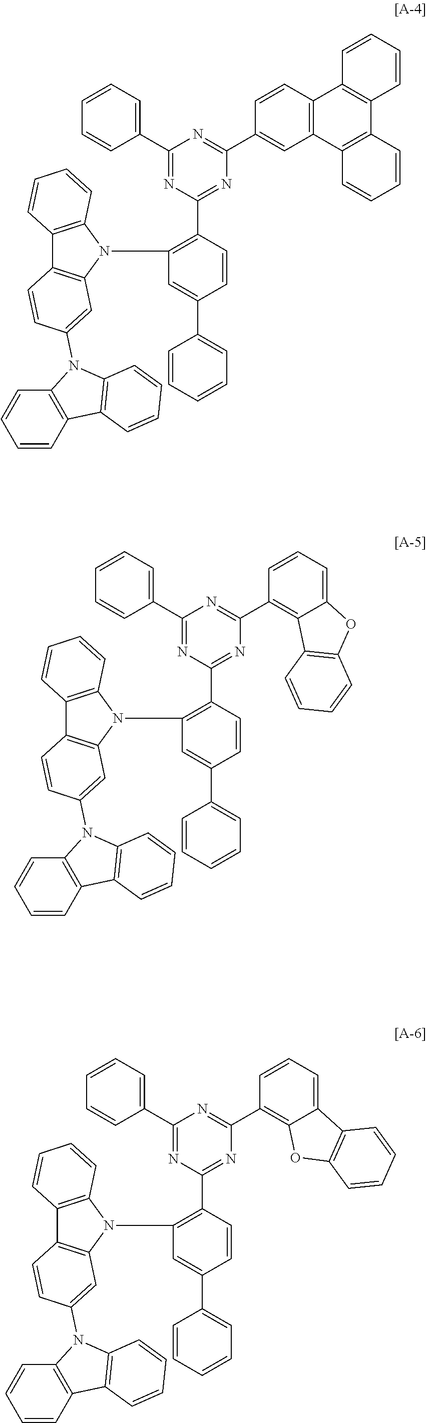 [SMILES: c1ccc(-c2ccc(-c3nc(-c4ccccc4)nc(-c4ccc5c6ccccc6c6ccccc6c5c4)n3)c(-n3c4ccccc4c4ccc(-n5c6ccccc6c6ccccc65)cc43)c2)cc1 ; c1ccc(-c2ccc(-c3nc(-c4ccccc4)nc(-c4cccc5oc6ccccc6c45)n3)c(-n3c4ccccc4c4ccc(-n5c6ccccc6c6ccccc65)cc43)c2)cc1 ; c1ccc(-c2ccc(-c3nc(-c4ccccc4)nc(-c4cccc5c4oc4ccccc45)n3)c(-n3c4ccccc4c4ccc(-n5c6ccccc6c6ccccc65)cc43)c2)cc1]

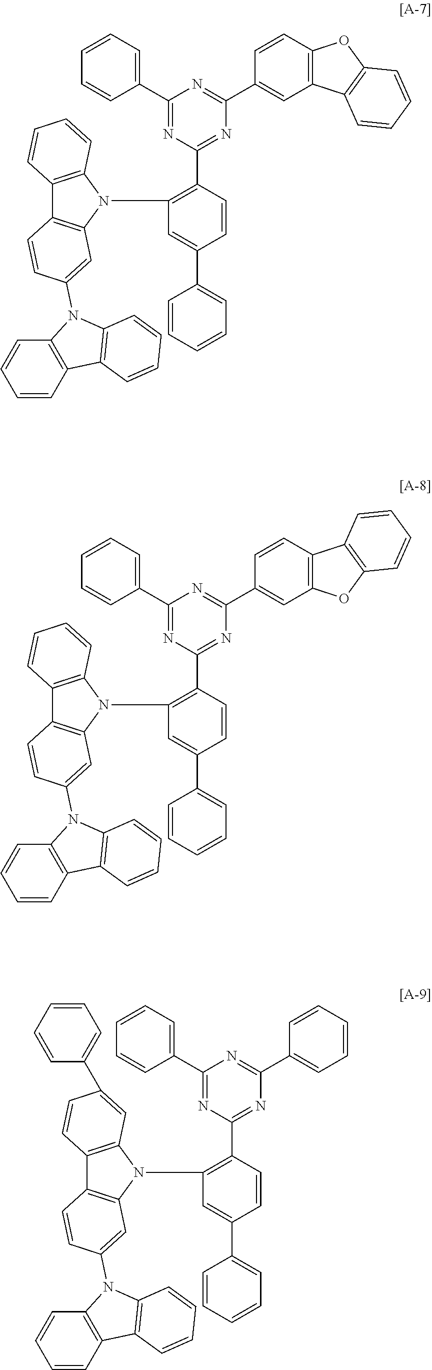 [SMILES: c1ccc(-c2ccc(-c3nc(-c4ccccc4)nc(-c4ccc5oc6ccccc6c5c4)n3)c(-n3c4ccccc4c4ccc(-n5c6ccccc6c6ccccc65)cc43)c2)cc1 ; c1ccc(-c2ccc(-c3nc(-c4ccccc4)nc(-c4ccc5c(c4)oc4ccccc45)n3)c(-n3c4ccccc4c4ccc(-n5c6ccccc6c6ccccc65)cc43)c2)cc1 ; c1ccc(-c2ccc(-c3nc(-c4ccccc4)nc(-c4ccccc4)n3)c(-n3c4cc(-c5ccccc5)ccc4c4ccc(-n5c6ccccc6c6ccccc65)cc43)c2)cc1]

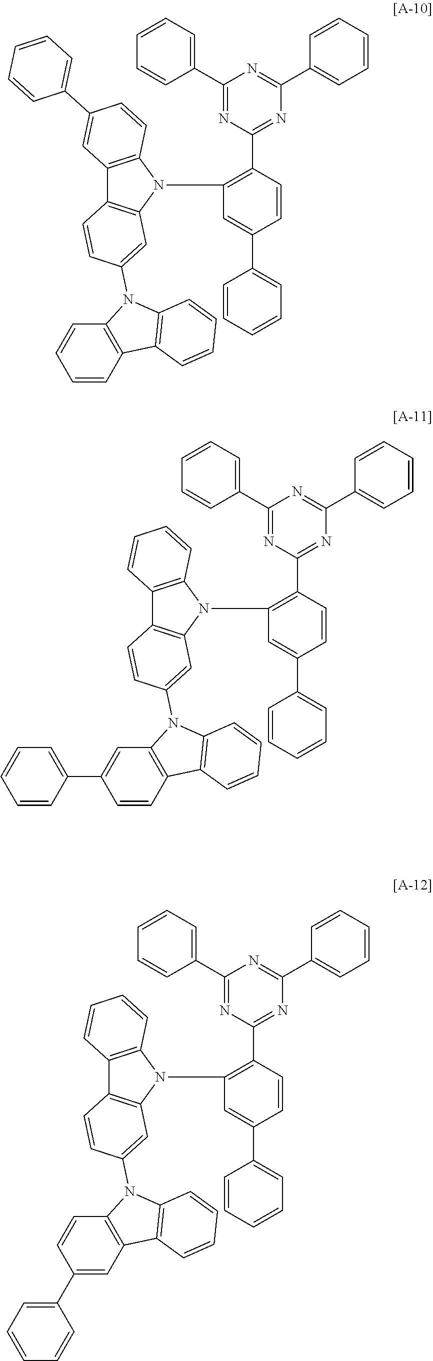 [SMILES: c1ccc(-c2ccc(-c3nc(-c4ccccc4)nc(-c4ccccc4)n3)c(-n3c4ccc(-c5ccccc5)cc4c4ccc(-n5c6ccccc6c6ccccc65)cc43)c2)cc1 ; c1ccc(-c2ccc(-c3nc(-c4ccccc4)nc(-c4ccccc4)n3)c(-n3c4ccccc4c4ccc(-n5c6ccccc6c6ccc(-c7ccccc7)cc65)cc43)c2)cc1 ; c1ccc(-c2ccc(-c3nc(-c4ccccc4)nc(-c4ccccc4)n3)c(-n3c4ccccc4c4ccc(-n5c6ccccc6c6cc(-c7ccccc7)ccc65)cc43)c2)cc1]

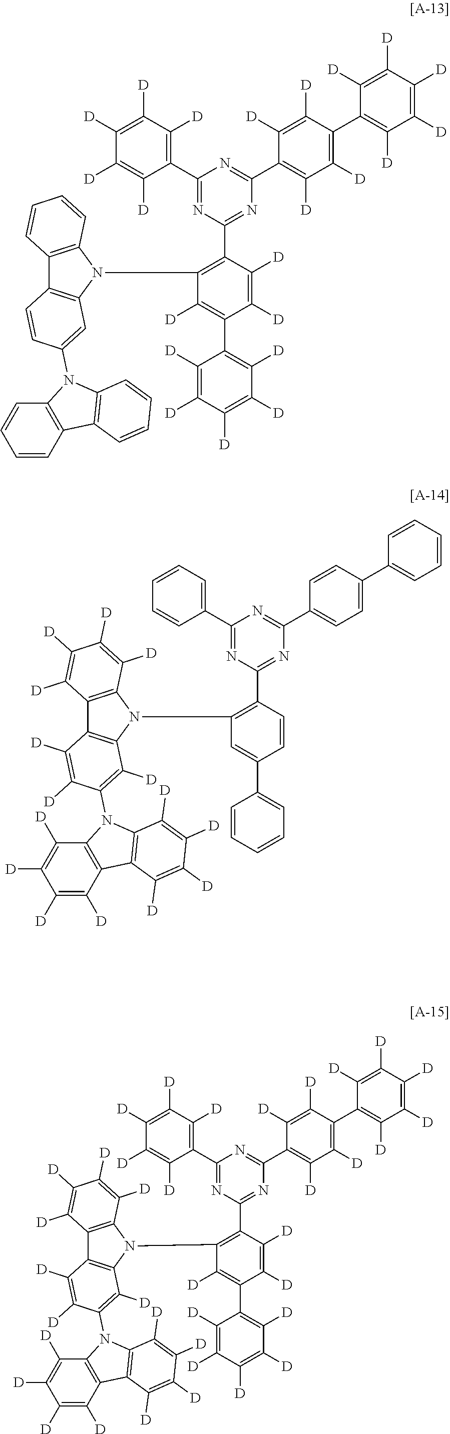

In addition to the aforementioned compound for an organic optoelectronic device, one or more compounds may be further included.

For example, a known hole transporting host may be further included as the second compound.

The first compound may be the aforementioned compound for an organic optoelectronic device, and the first compound and the second compound may be included in a weight ratio of, for example, 1:99 to 99:1. Within the above range, efficiency and life-span can be improved by implementing bipolar characteristics by adjusting the appropriate weight ratio using the electron transport ability of the first compound and the hole transport ability of the second compound. Within the above range, they may be included in a weight ratio of, for example, about 10:90 to 90:10, about 20:80 to 80:20, for example, about 20:80 to about 70:30, about 20:80 to about 60:40, or about 20:80 to about 50:50. As a specific example, they may be included in a weight ratio of 20:80, 30:70, or 40:60.

In addition to the aforementioned first and second compounds, one or more compounds may be further included.

For example, a dopant may be further included.

The dopant may be, for example, a phosphorescent dopant, such as a red, green, or blue phosphorescent dopant, and may be, for example, a red or green phosphorescent dopant.

The dopant is a material mixed with the compound for an organic optoelectronic device in a small amount to cause light emission and may be generally a material such as a metal complex that emits light by multiple excitation into a triplet or more. The dopant may be, for example an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

### [Chemical Formula Z]

L³MX

In Chemical Formula Z, M is a metal, and L³ and X are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof, and L³ and X may be, for example a bidentate ligand.

Examples of the ligands represented by L³ and X may be selected from the chemical formulas listed in Group A, but are not limited thereto.

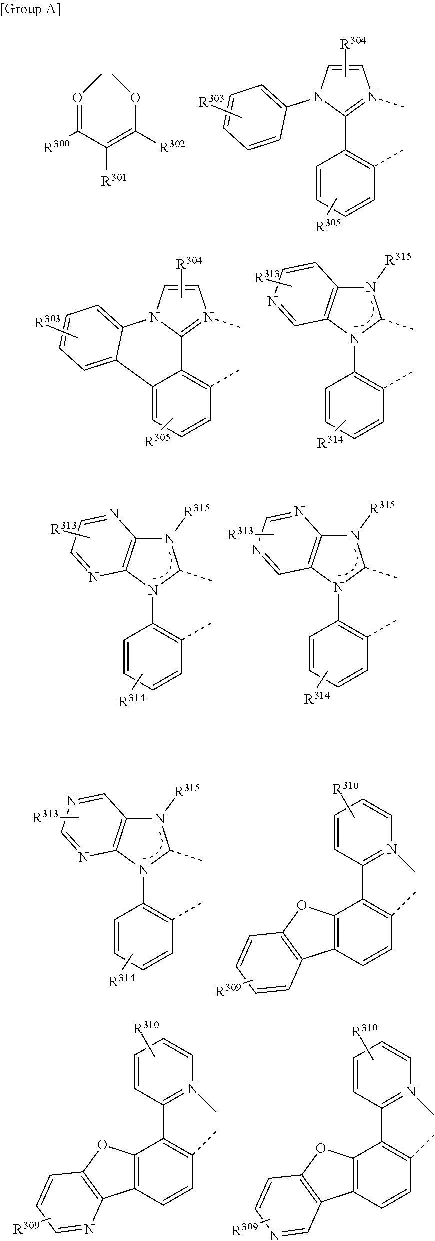

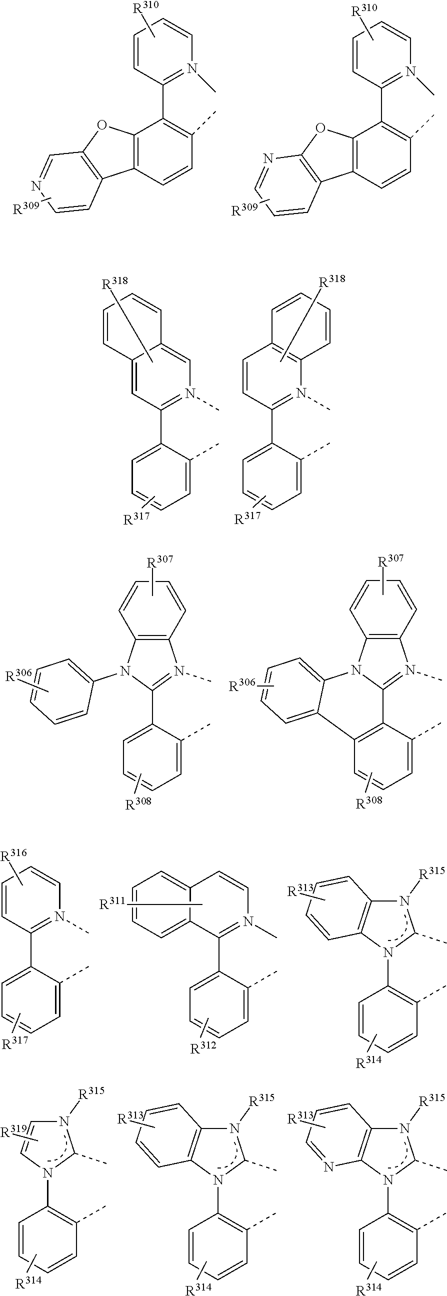

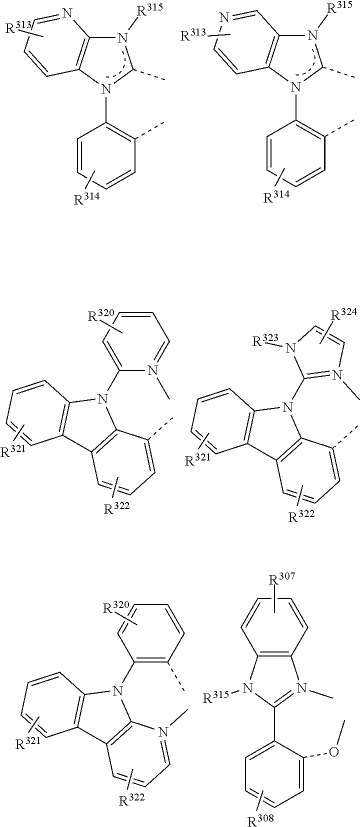

In Group A,
R³⁰⁰ to R³⁰² are each independently hygrogen deuterium to C1 to C30 alkyl group that is substituted or unsubstituted with a halogen, a C6 to C30 aryl group that is substituted or unsubstituted with a C1 to C30 alkyl, or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

For example, a dopant represented by Chemical Formula V may be included.

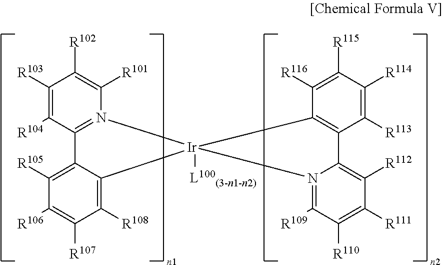

In Chemical Formula V,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴
R¹³² to R¹³⁴ are each independently a C1 to C6 alkyl group,
at least one of R¹⁰¹ to R¹¹⁶ is a functional group represented by Chemical Formula V-1,
L¹⁰⁰ is a bidentate ligand of a monovalent anion, and is a ligand that coordinates to iridium through a lone pair of carbons or heteroatoms, and
n¹ and n² are each independently any one of integers of 0 to 3, and n1+n2 is any one of integers of 1 to 3,

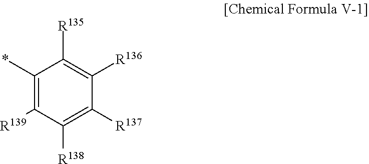

wherein, in Chemical Formula V-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* means a portion linked to a carbon atom.

For example, the dopant represented by Chemical Formula Z-1 may be included.

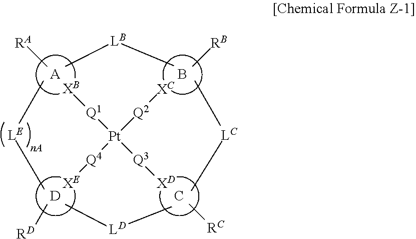

In Chemical Formula Z-1, rings A, B, C, and D are each independently a 5-membered or 6-membered carbocyclic or heterocyclic ring;
R^{A}, R^{B}, R^{C}, and R^{D} are each independently mono-, di-, tri-, or tetra-substitution, or unsubstitution;
L^{B}, L^{C}, and L^{D} are each independently a direct bond, BR, NR, PR, O, S, Se, C═O, S═O, SO₂, CRR′, SiRR′, GeRR′, or a combination thereof,
when nA is 1, L^{E} may be a direct bond, BR, NR, PR, O, S, Se, C═O, S═O, SO₂, CRR′, SiRR′, GeRR′, or a combination thereof; and when nA is 0, L^{E} does not exist;
R^{A}, R^{B}, R^{C}, R^{D}, R, and R′ are each independently hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ are optionally linked to each other to provide a ring; X^{B}, X^{c}, X^{D}, and X^{E} are each independently selected from carbon and nitrogen; and Q¹, Q², Q³, and Q⁴ each represent oxygen or a direct bond.

The dopant according to an embodiment may be a platinum complex, and may be represented by Chemical Formula VI.

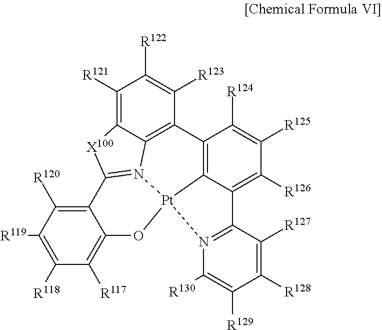

In Chemical Formula VI,
X¹⁰⁰ is selected from O, S, and NR¹³¹,
R¹¹⁷ to R¹³¹ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a C1 to C6 alkyl group, and
at least one of R¹¹⁷ to R¹³¹ is —SiR¹³²R¹³³R¹³⁴ or a tert-butyl group.

Hereinafter, an organic optoelectronic device including the aforementioned compound for an organic optoelectronic device or composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

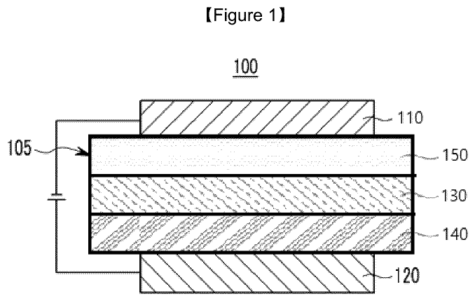

FIG. 1 is a cross-sectional view showing an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer.

The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, and BaF₂/Ca, but is not limited thereto.

The organic layer 105 may include the aforementioned compound for an organic optoelectronic device.

The organic layer 105 may include a light emitting layer 130 and the light emitting layer 130 may include the aforementioned compound for an organic optoelectronic device.

The composition for an organic optoelectronic device further including a dopant may be, for example, a red or green light emitting composition.

The light emitting layer 130 may include, for example, the aforementioned compound for an organic optoelectronic device, as a phosphorescent host.

The organic layer may further include a charge transport region in addition to the light emitting layer.

The charge transport region may be, for example, a hole transport region 140.

The hole transport region 140 may further increase hole injection and/or hole mobility between the anode 120 and the light emitting layer 130 and block electrons.

Specifically, the hole transport region 140 may include a hole transport layer between the anode 120 and the light emitting layer 130, and a hole transport auxiliary layer between the light emitting layer 130 and the hole transport layer, and at least one of the compounds of Group B may be included in at least one of the hole transport layer and the hole transport auxiliary layer.

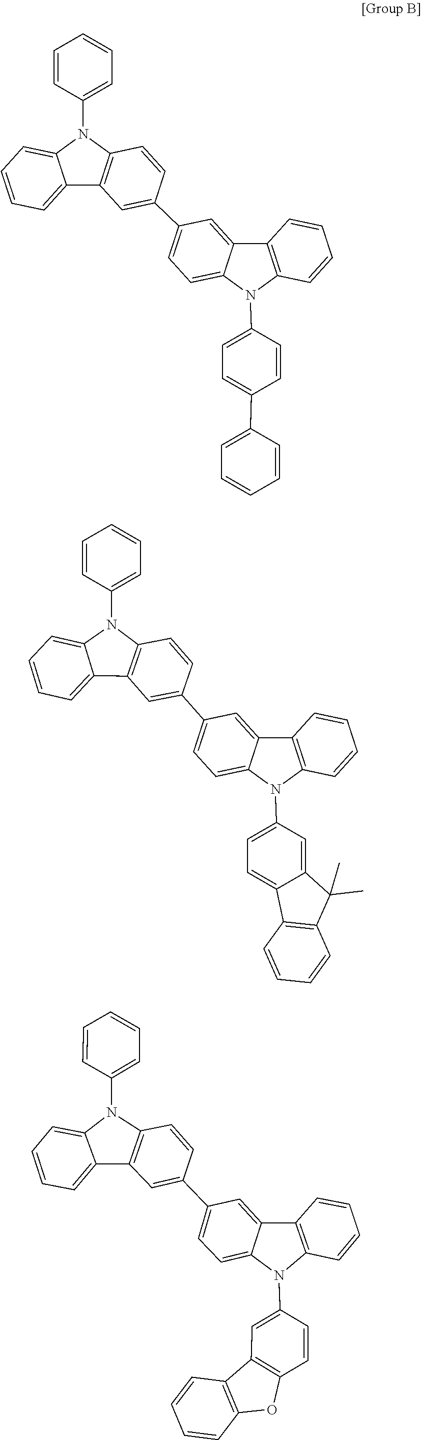 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

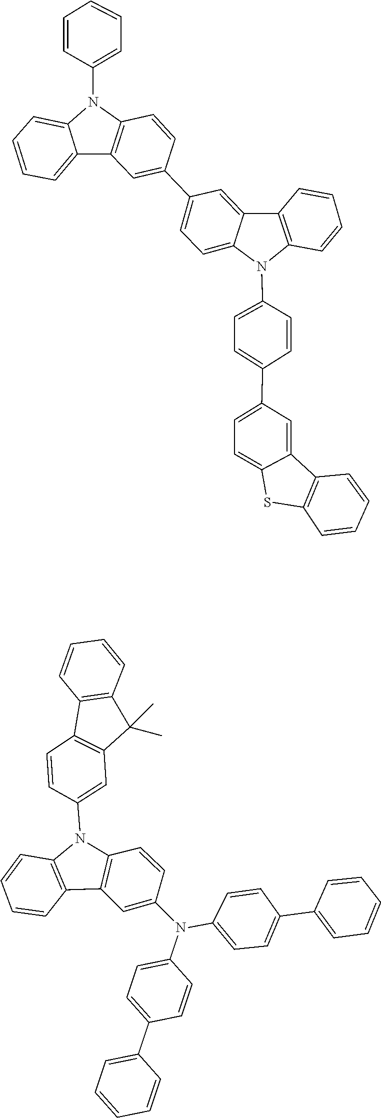

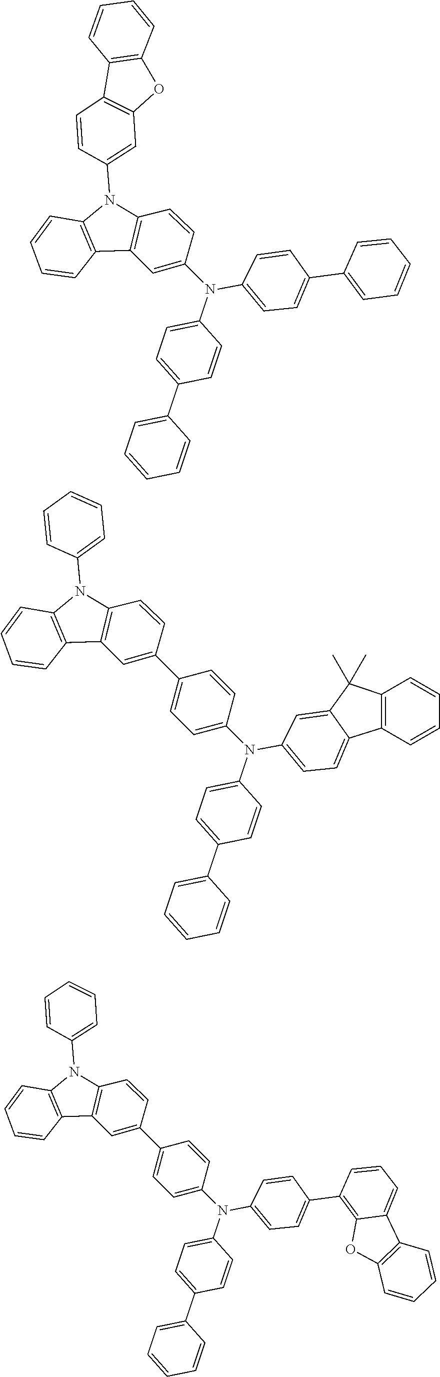

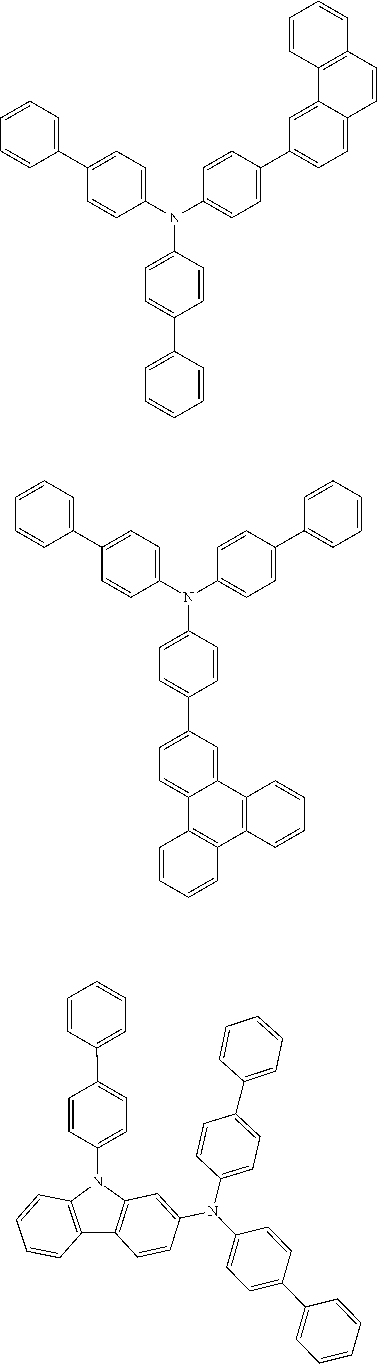

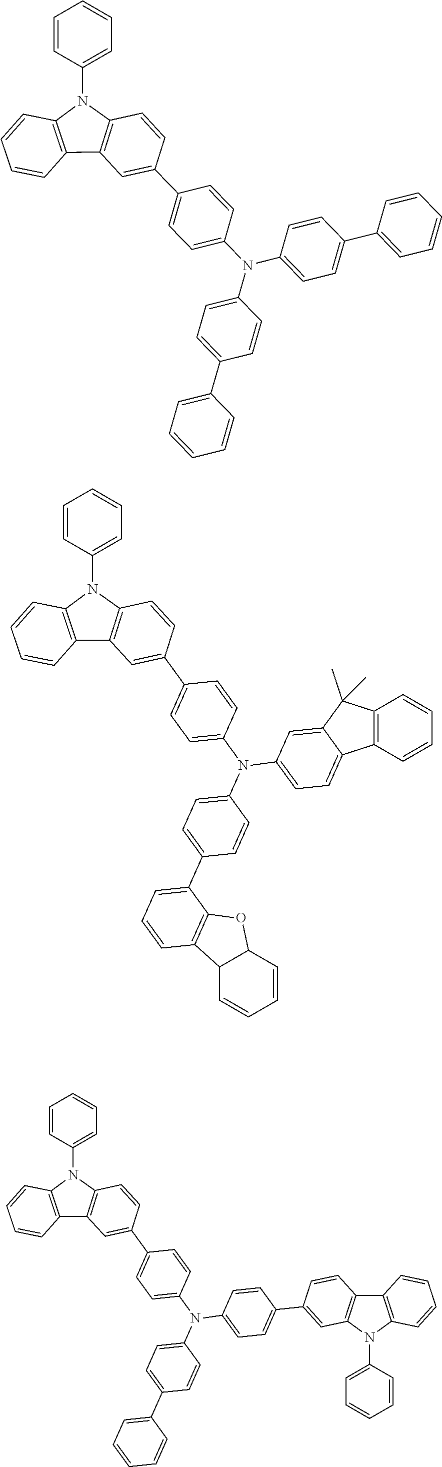 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4OC4C=CC=CC54)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

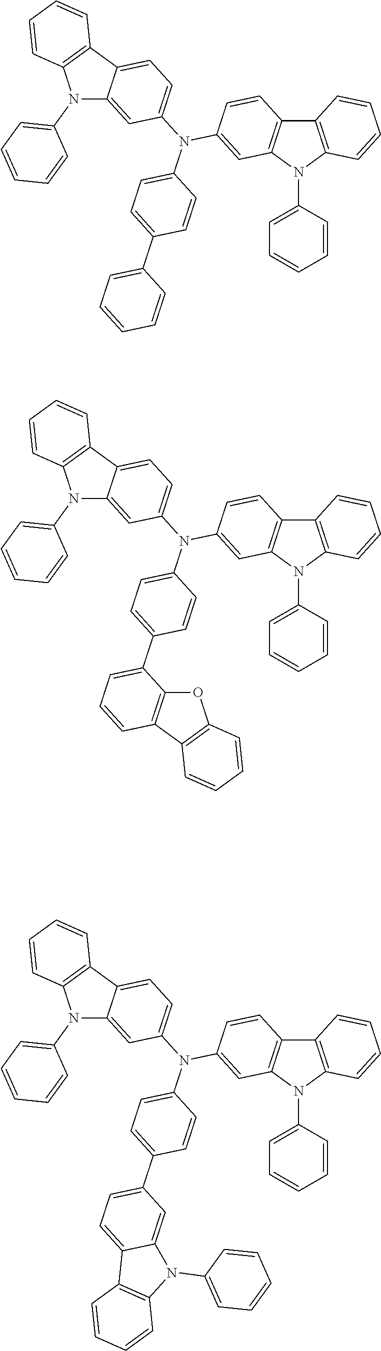 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

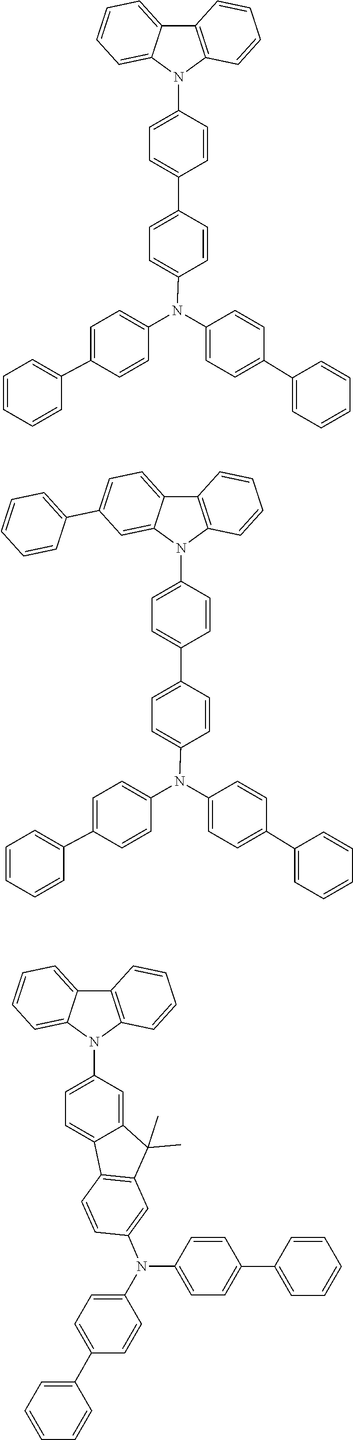 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21]

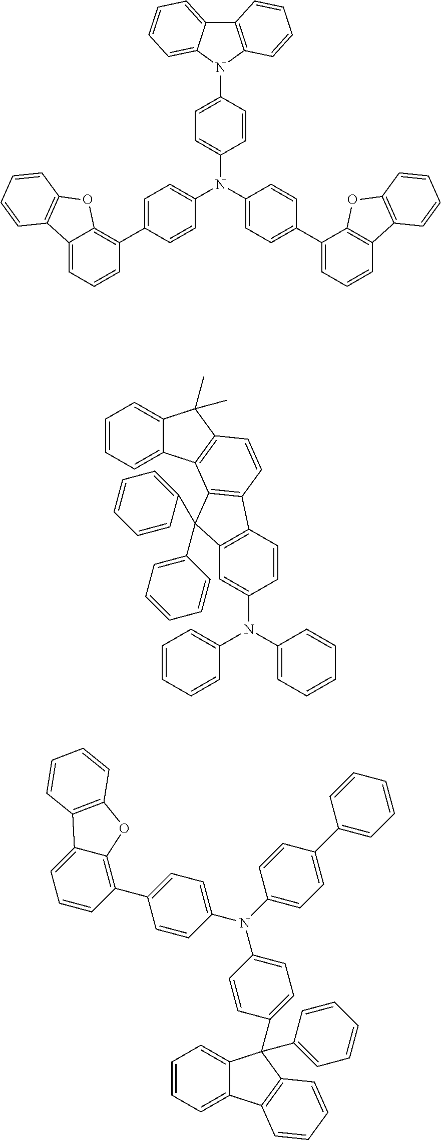 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1]

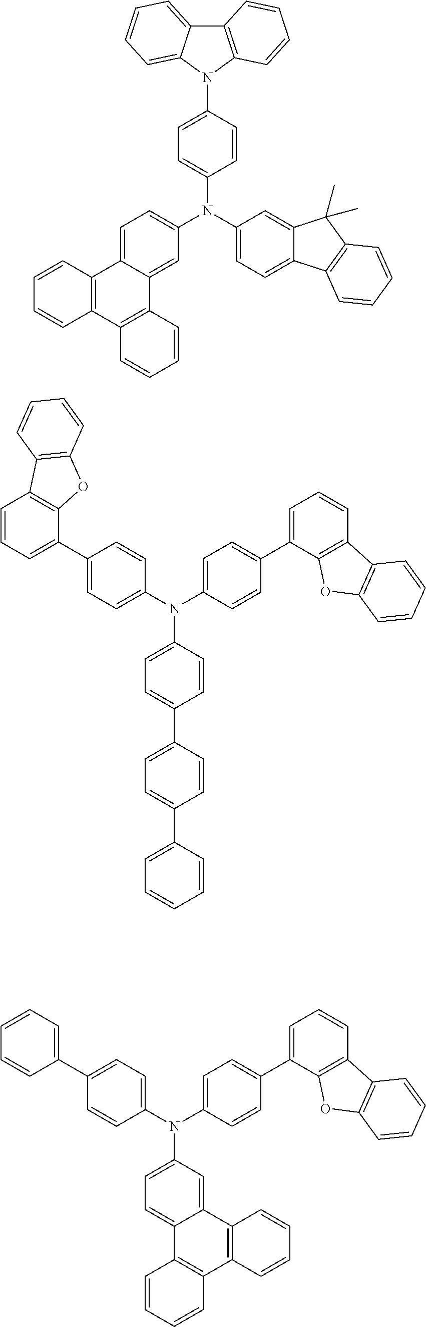 [SMILES: CC1(C)c2ccccc2-c2ccc(N(c3ccc(-n4c5ccccc5c5ccccc54)cc3)c3ccc4c5ccccc5c5ccccc5c4c3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1]

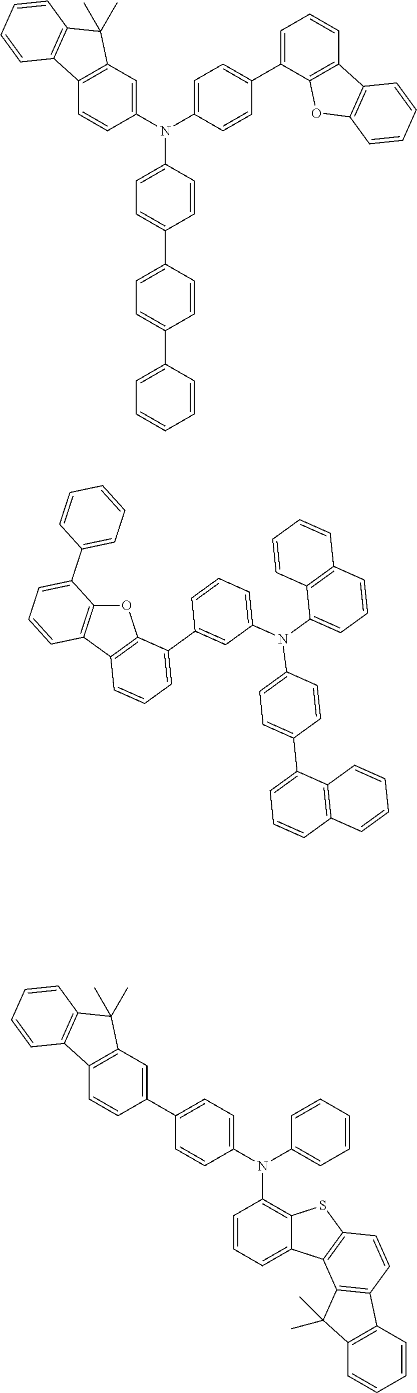 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21]

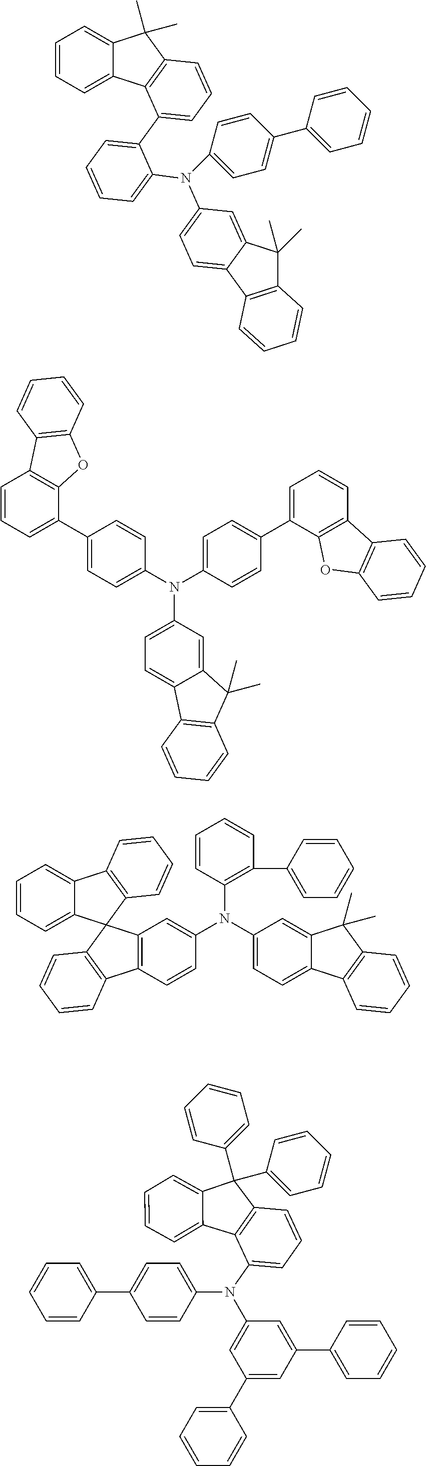

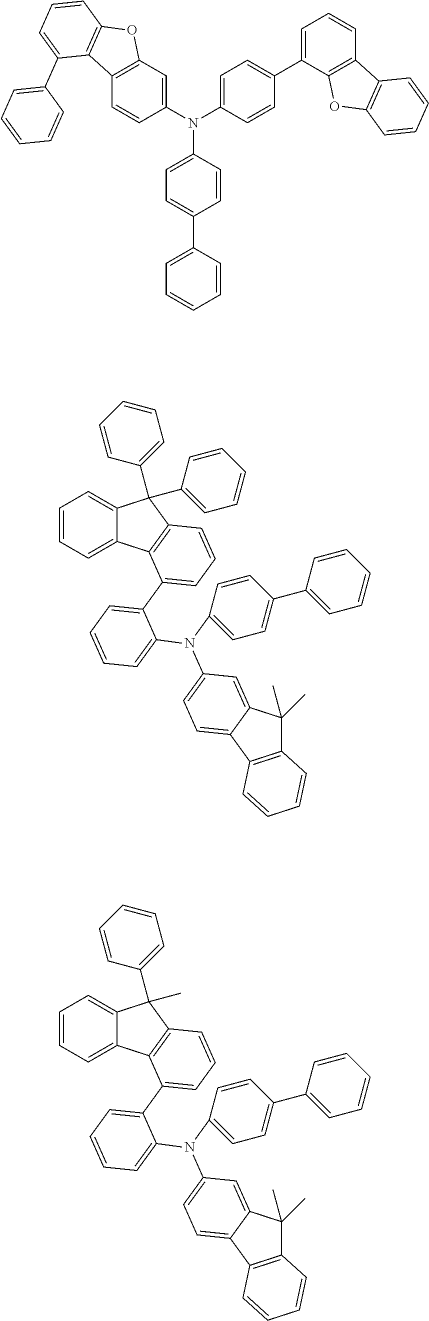 [SMILES: c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21]

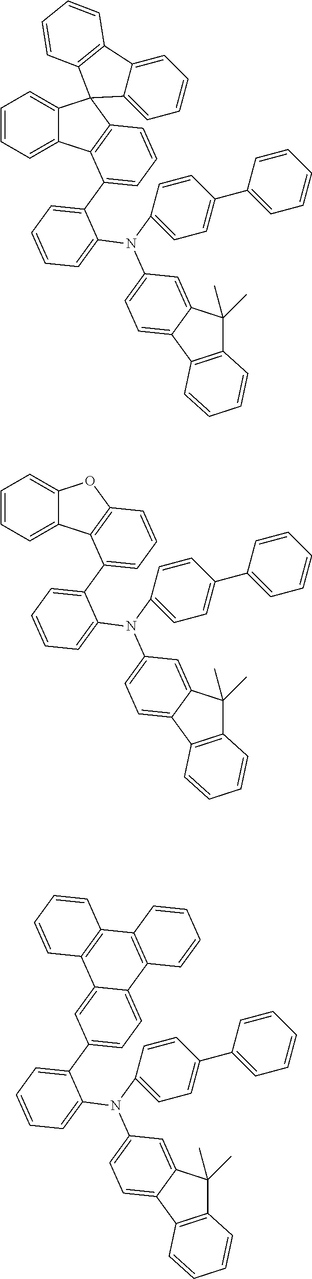 [SMILES: CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21 ; CC1(C)c2ccccc2-c2ccc(N(c3ccc(-c4ccccc4)cc3)c3ccccc3-c3ccc4c5ccccc5c5ccccc5c4c3)cc21]

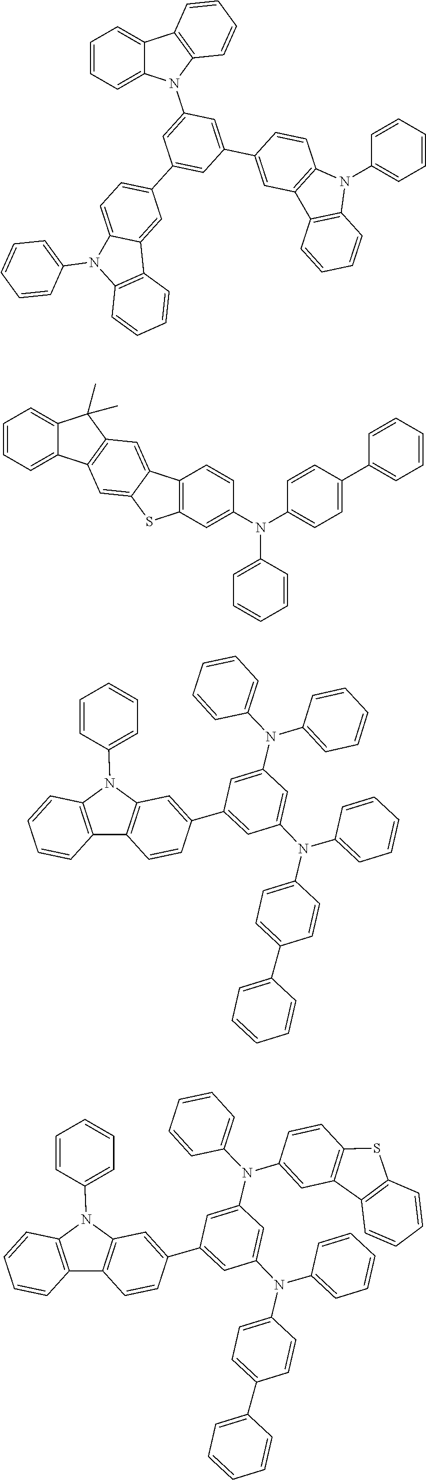

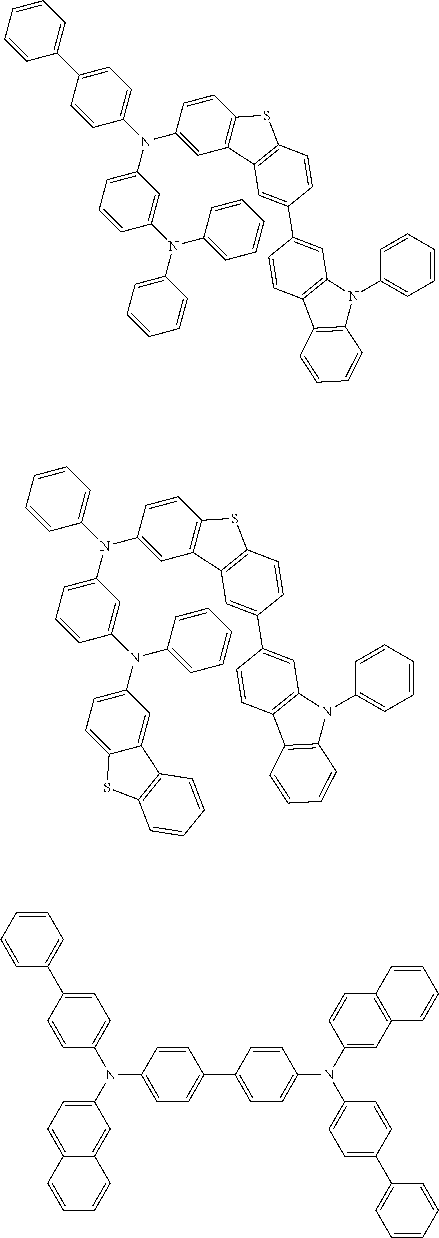 [SMILES: c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1]

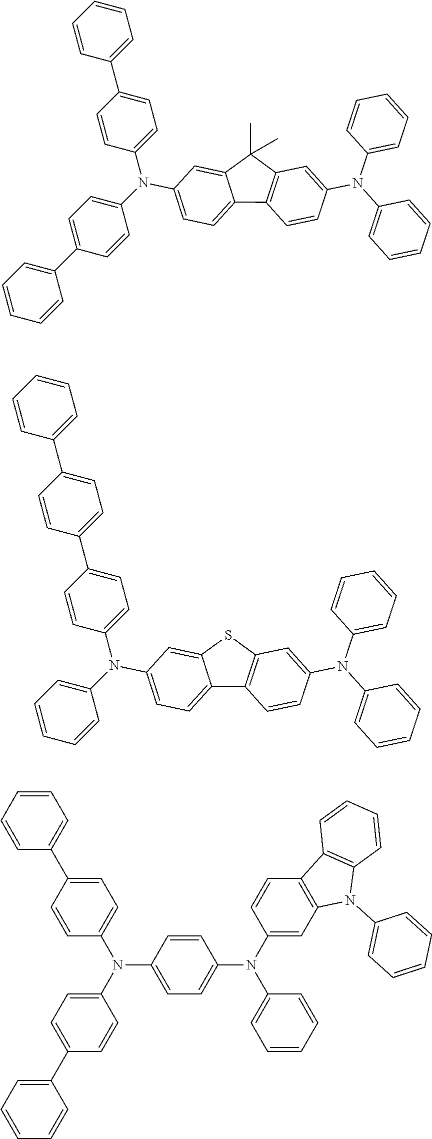 [SMILES: CC1(C)c2cc(N(c3ccccc3)c3ccccc3)ccc2-c2ccc(N(c3ccc(-c4ccccc4)cc3)c3ccc(-c4ccccc4)cc3)cc21 ; c1ccc(-c2ccc(-c3ccc(N(c4ccccc4)c4ccc5c(c4)sc4cc(N(c6ccccc6)c6ccccc6)ccc45)cc3)cc2)cc1 ; c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1]

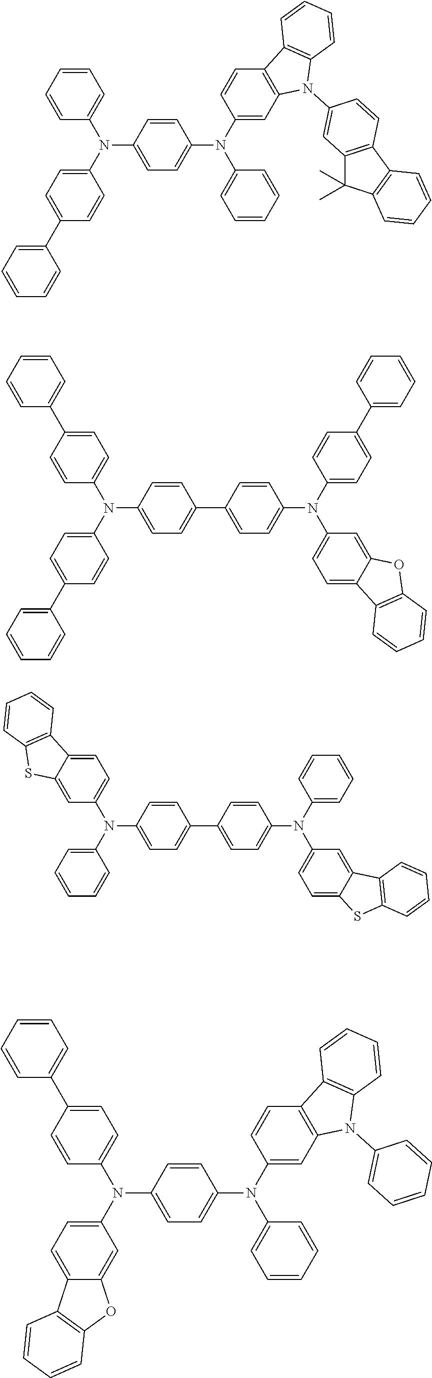

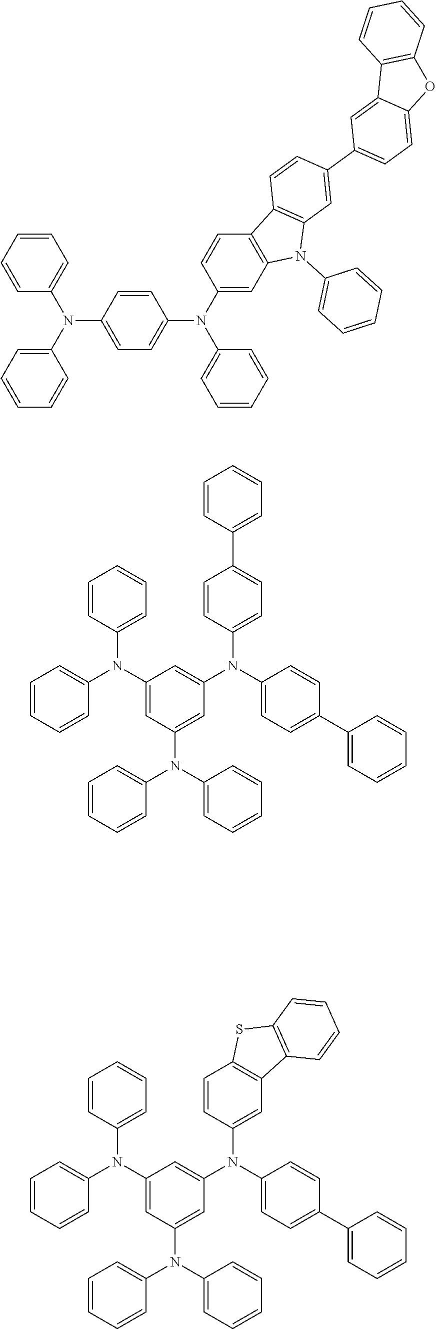 [SMILES: c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1]

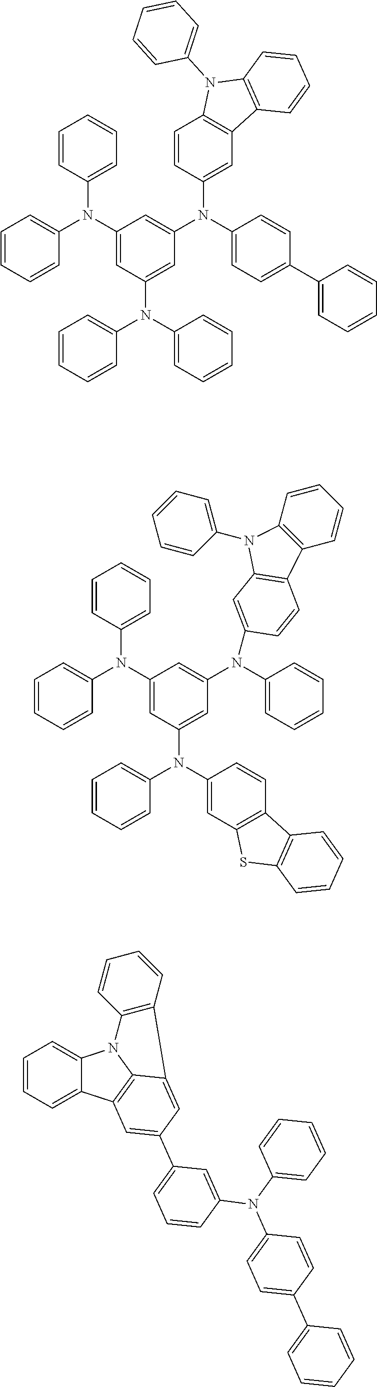 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1]

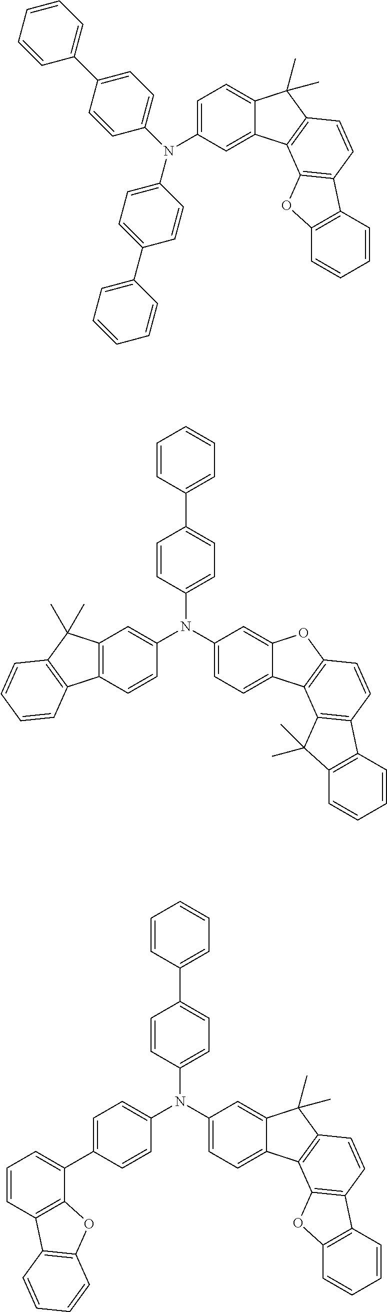 [SMILES: CC1(C)c2ccc(N(c3ccc(-c4ccccc4)cc3)c3ccc(-c4ccccc4)cc3)cc2-c2c1ccc1c2oc2ccccc21 ; CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21]

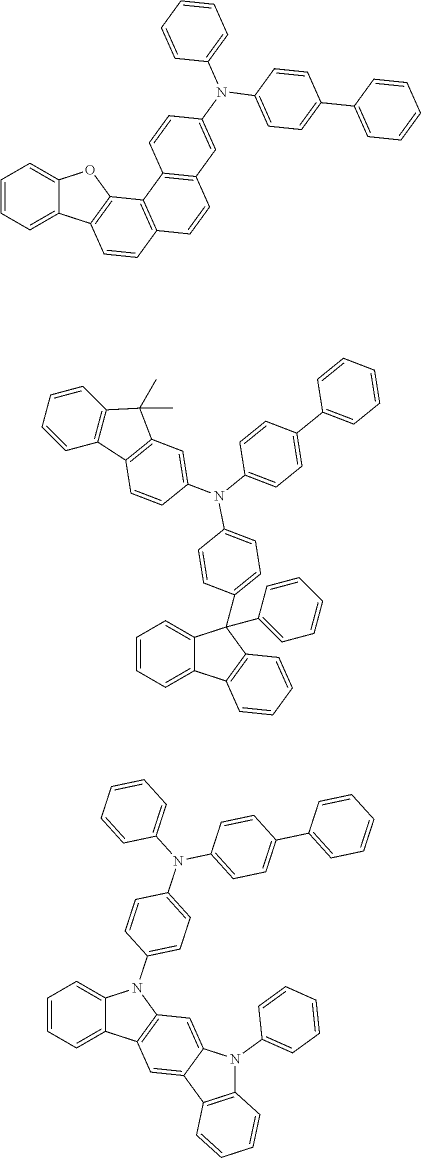 [SMILES: c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1]

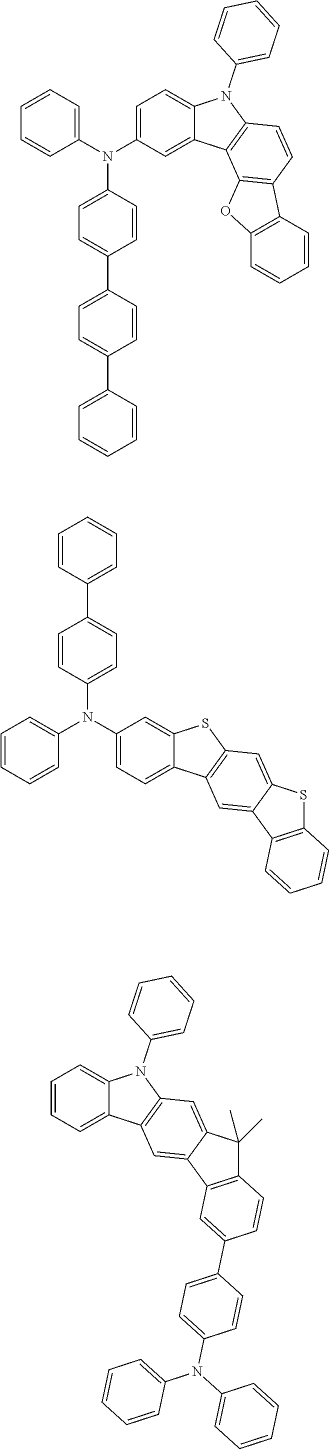 [SMILES: c1ccc(-c2ccc(-c3ccc(N(c4ccccc4)c4ccc5c(c4)c4c6oc7ccccc7c6ccc4n5-c4ccccc4)cc3)cc2)cc1 ; c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21]

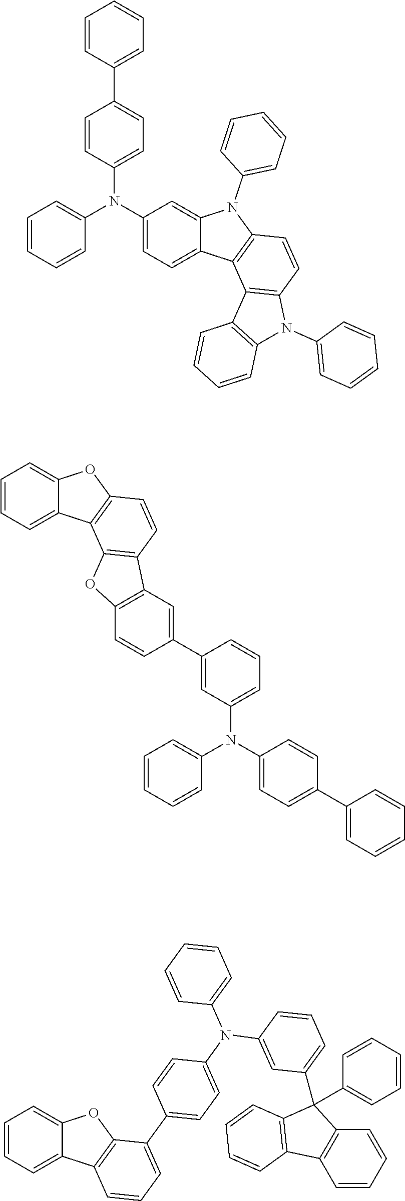 [SMILES: c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1]

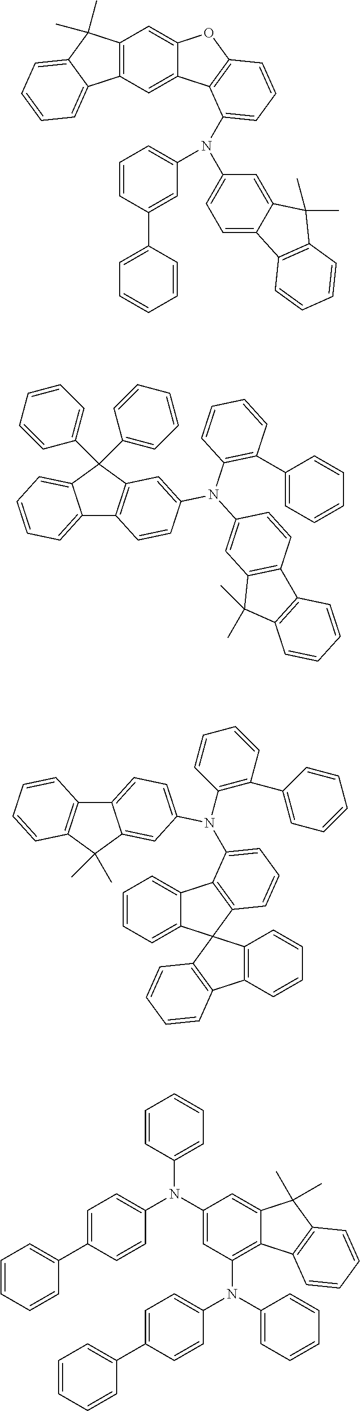 [SMILES: CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2c(N(c3ccccc3)c3ccc(-c4ccccc4)cc3)cc(N(c3ccccc3)c3ccc(-c4ccccc4)cc3)cc21]

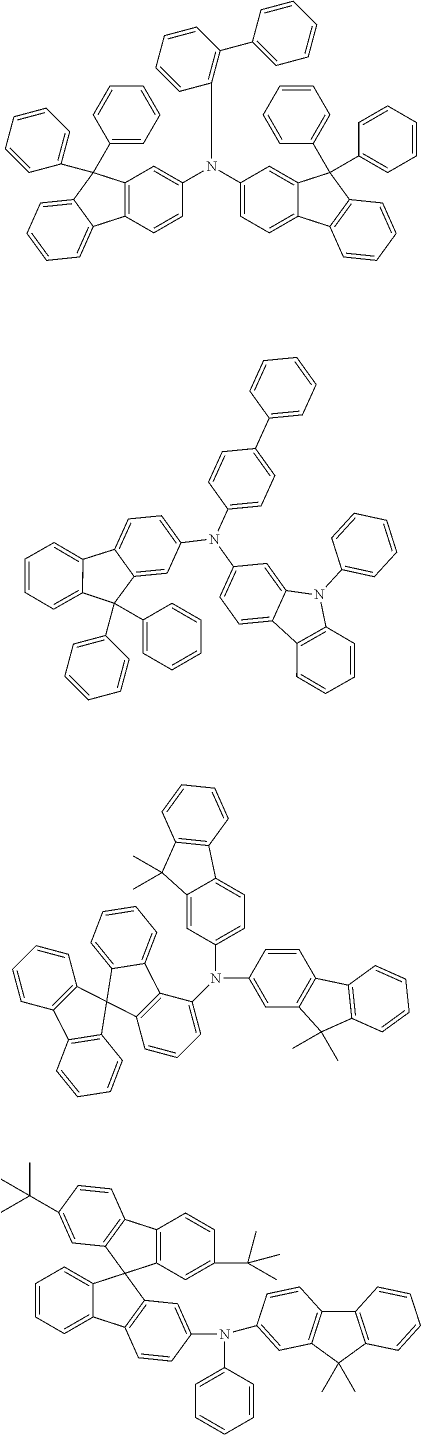 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

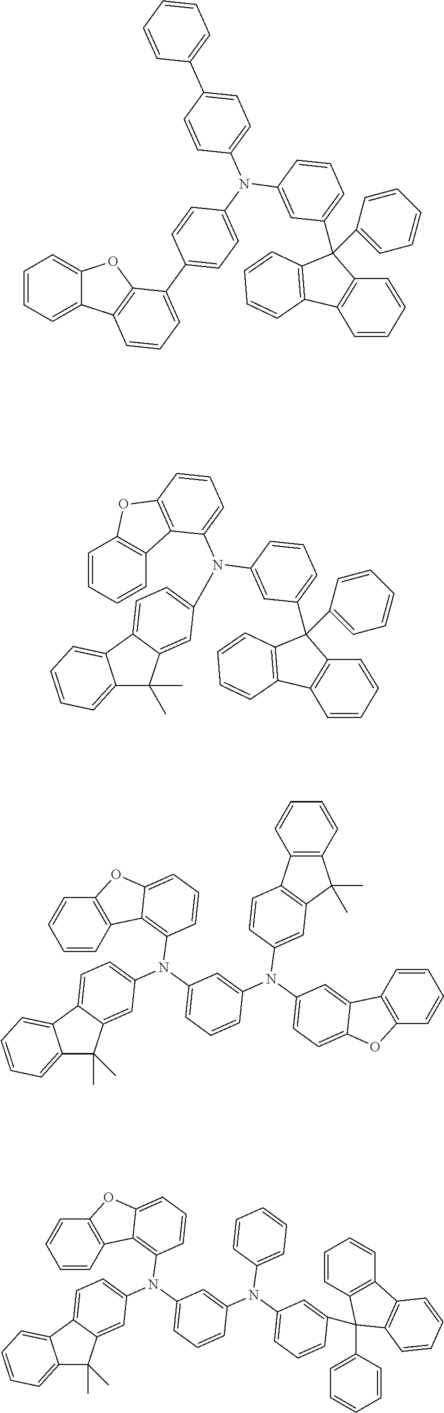 [SMILES: c1ccc(-c2ccc(N(c3ccc(-c4cccc5c4oc4ccccc45)cc3)c3cccc(C4(c5ccccc5)c5ccccc5-c5ccccc54)c3)cc2)cc1 ; CC1(C)c2ccccc2-c2ccc(N(c3cccc(C4(c5ccccc5)c5ccccc5-c5ccccc54)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21]

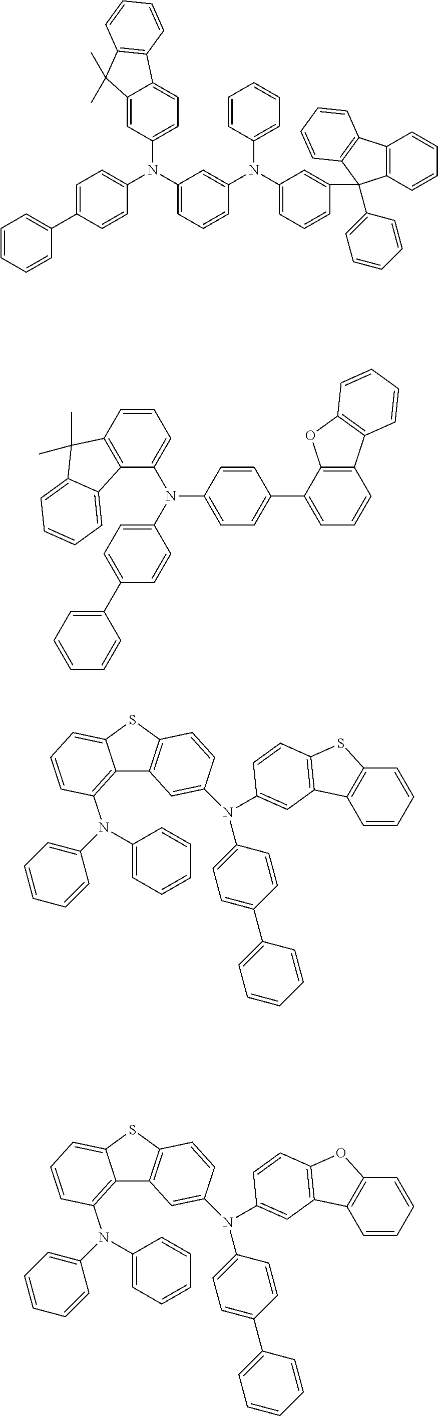 [SMILES: CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1]

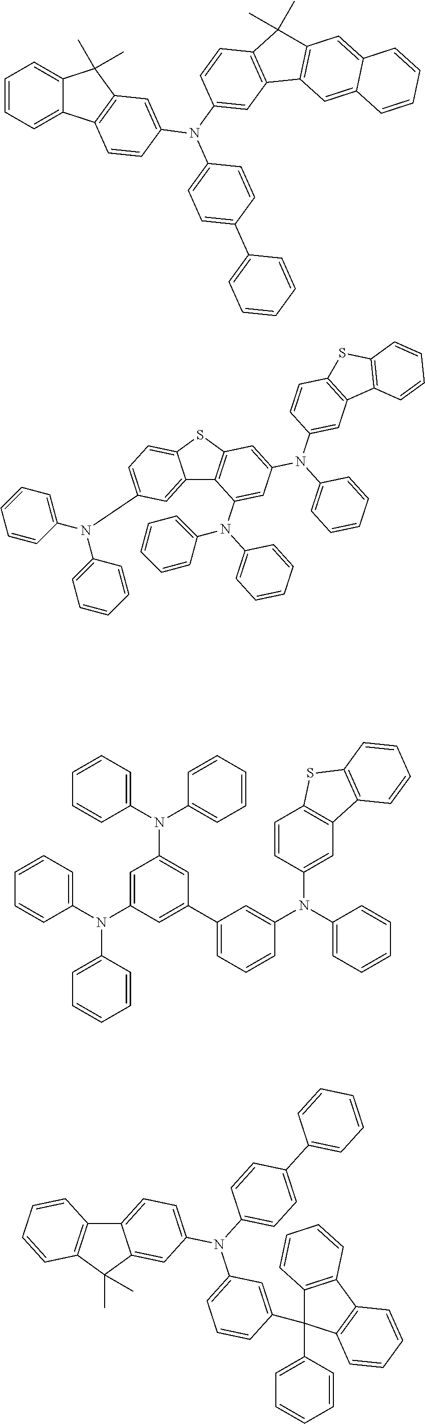 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In the hole transport region, in addition to the compounds described above, known compounds disclosed in U.S. Pat. No. 5,061,569A, JP1993-009471A, W01995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

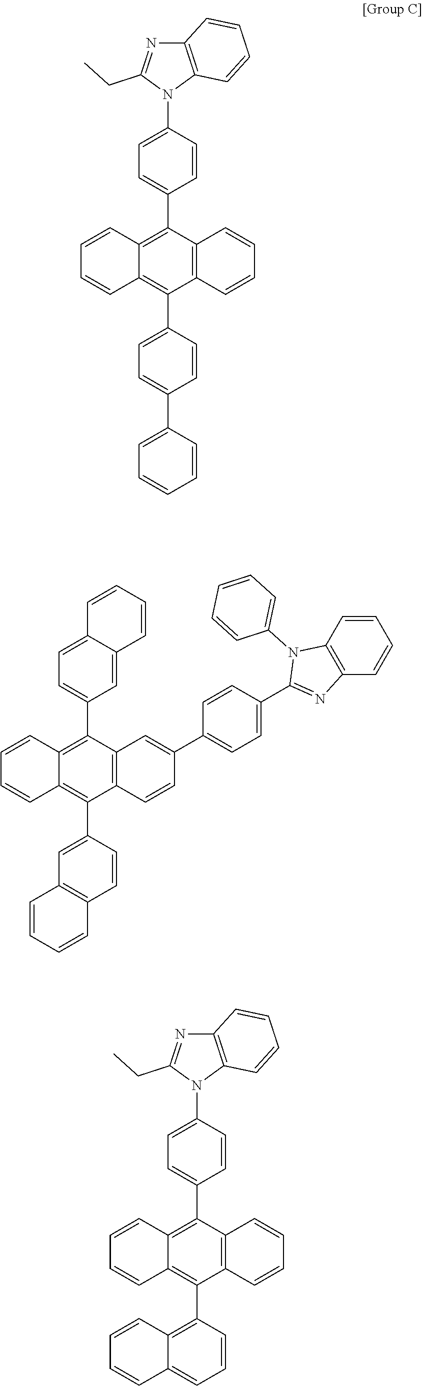 [SMILES: CCc1nc2ccccc2n1-c1ccc(-c2c3ccccc3c(-c3ccc(-c4ccccc4)cc3)c3ccccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CCc1nc2ccccc2n1-c1ccc(-c2c3ccccc3c(-c3cccc4ccccc34)c3ccccc23)cc1]

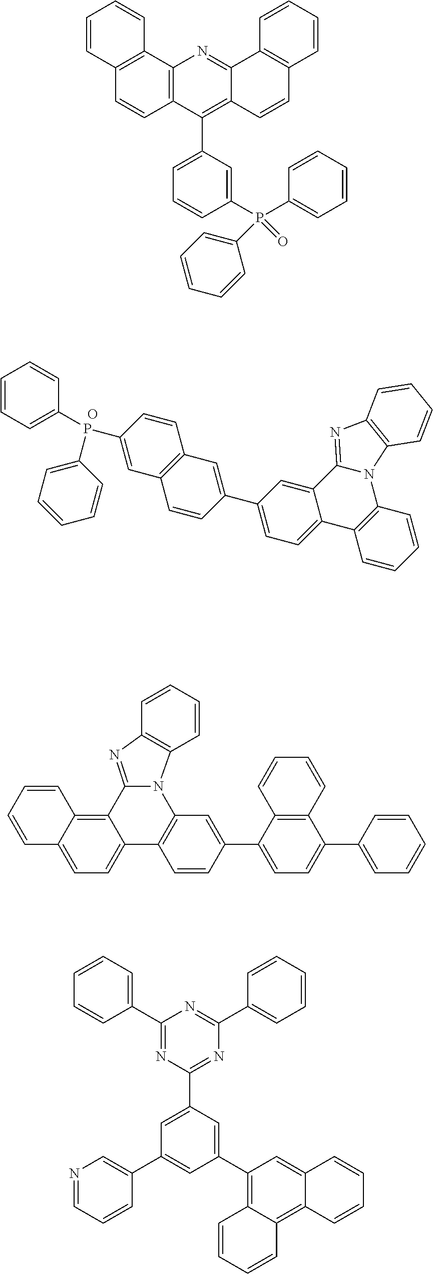

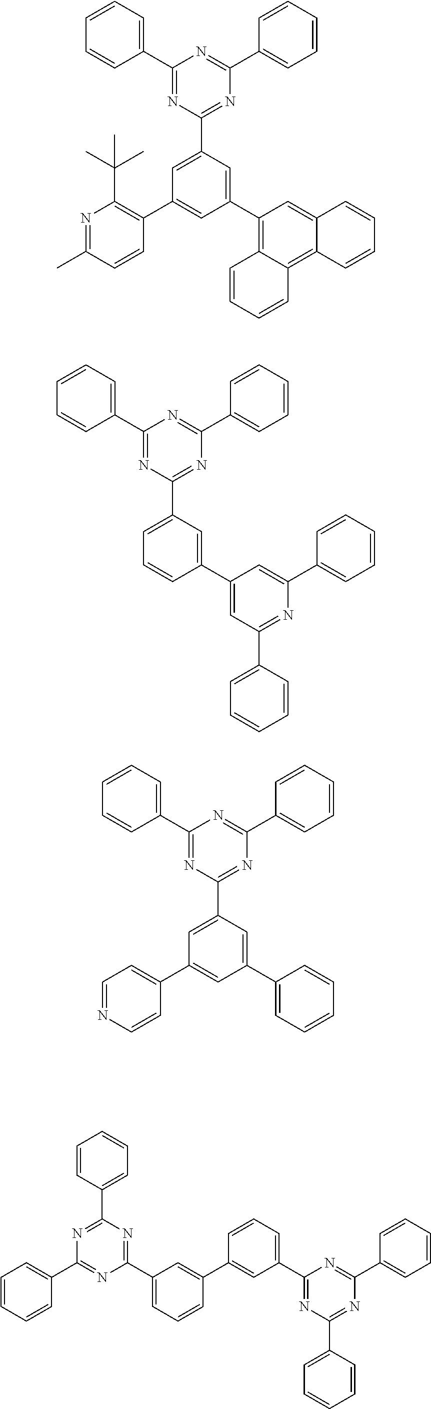 [SMILES: Cc1ccc(-c2cc(-c3nc(-c4ccccc4)nc(-c4ccccc4)n3)cc(-c3cc4ccccc4c4ccccc34)c2)c(C(C)(C)C)n1 ; c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1]

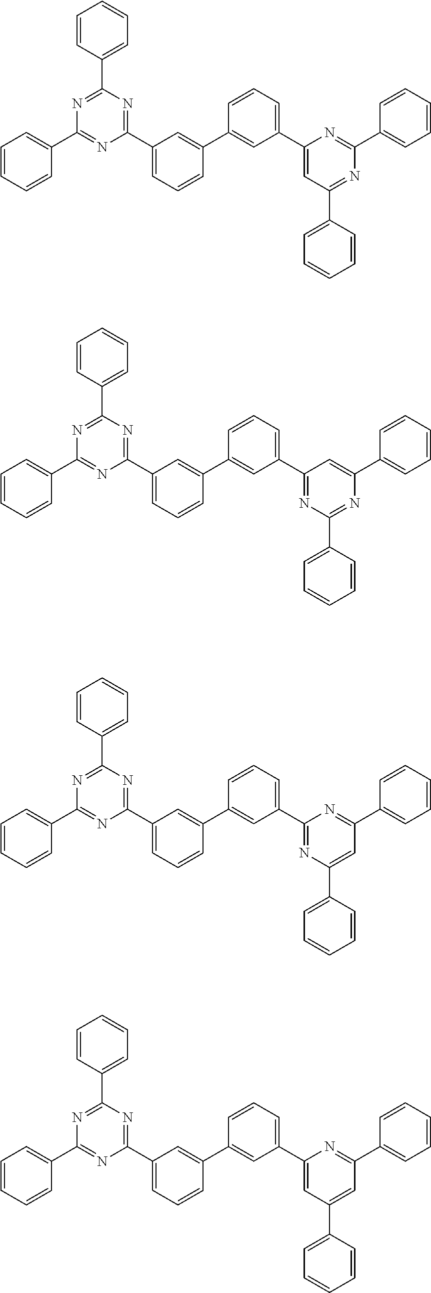 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1]

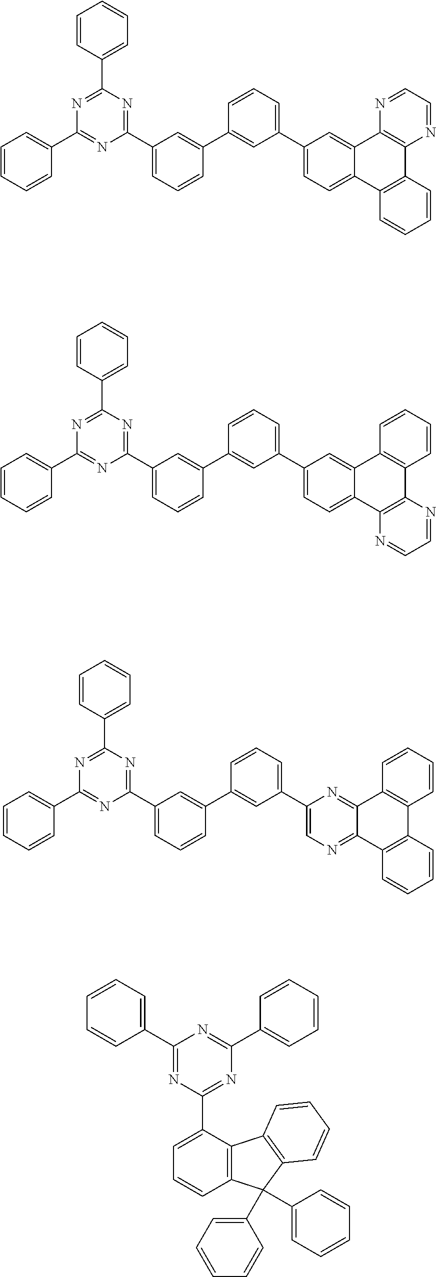 [SMILES: c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1 ; c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1]

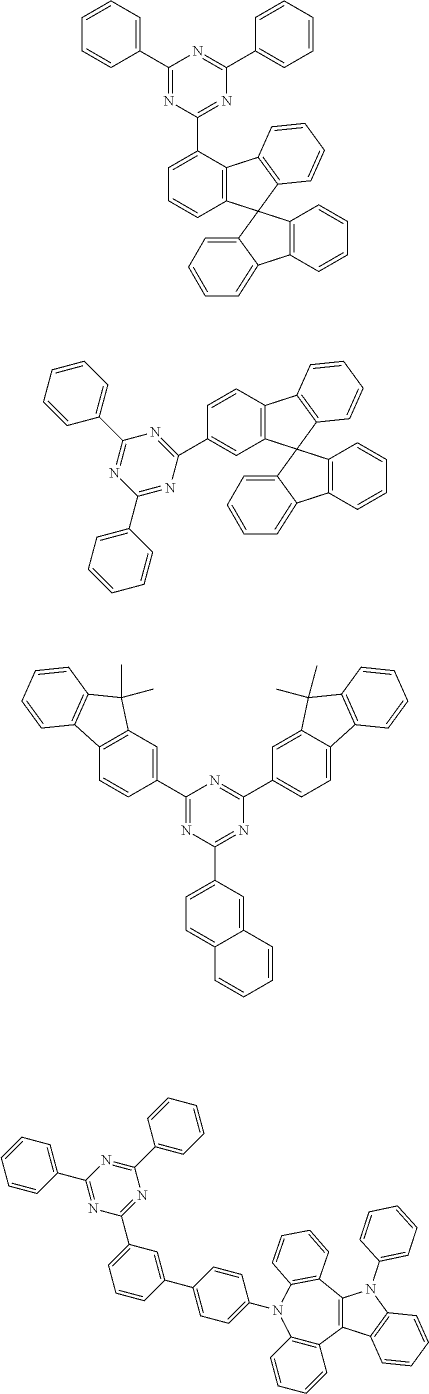 [SMILES: c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1 ; CC1(C)c2ccccc2-c2ccc(-c3nc(-c4ccc5c(c4)C(C)(C)c4ccccc4-5)nc(-c4ccc5ccccc5c4)n3)cc21 ; c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1]

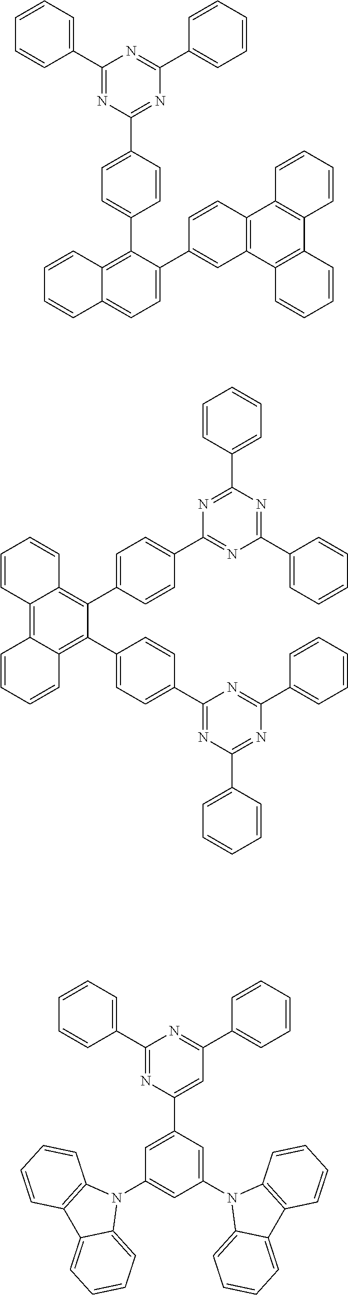 [SMILES: c1ccc(-c2nc(-c3ccccc3)nc(-c3ccc(-c4c(-c5ccc6c7ccccc7c7ccccc7c6c5)ccc5ccccc45)cc3)n2)cc1 ; c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1 ; c1ccc(-c2cc(-c3cc(-n4c5ccccc5c5ccccc54)cc(-n4c5ccccc5c5ccccc54)c3)nc(-c3ccccc3)n2)cc1]

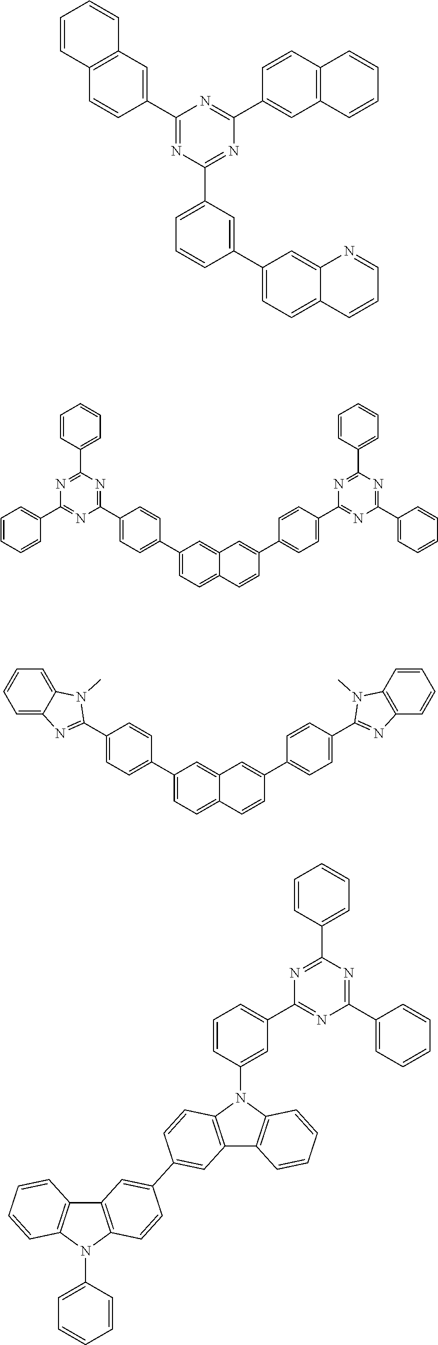 [SMILES: c1cc(-c2ccc3cccnc3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

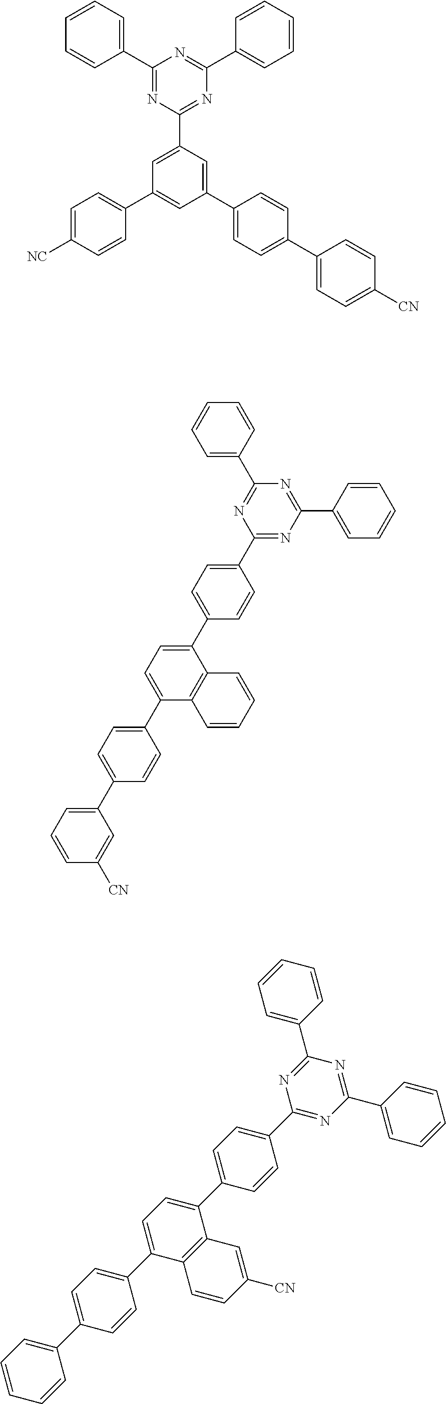 [SMILES: N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

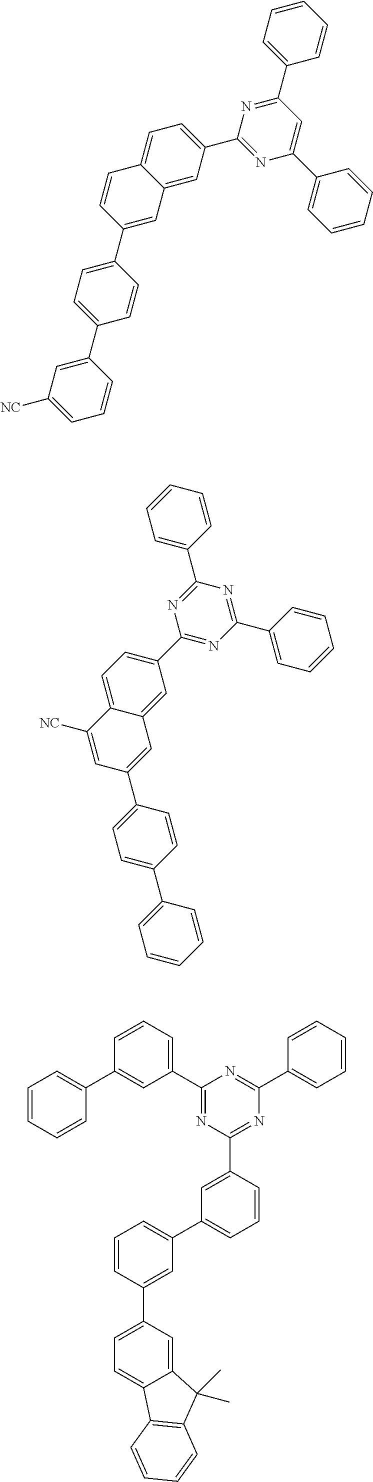 [SMILES: N#Cc1cccc(-c2ccc(-c3ccc4ccc(-c5nc(-c6ccccc6)cc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12 ; CC1(C)c2ccccc2-c2ccc(-c3cccc(-c4cccc(-c5nc(-c6ccccc6)nc(-c6cccc(-c7ccccc7)c6)n5)c4)c3)cc21]

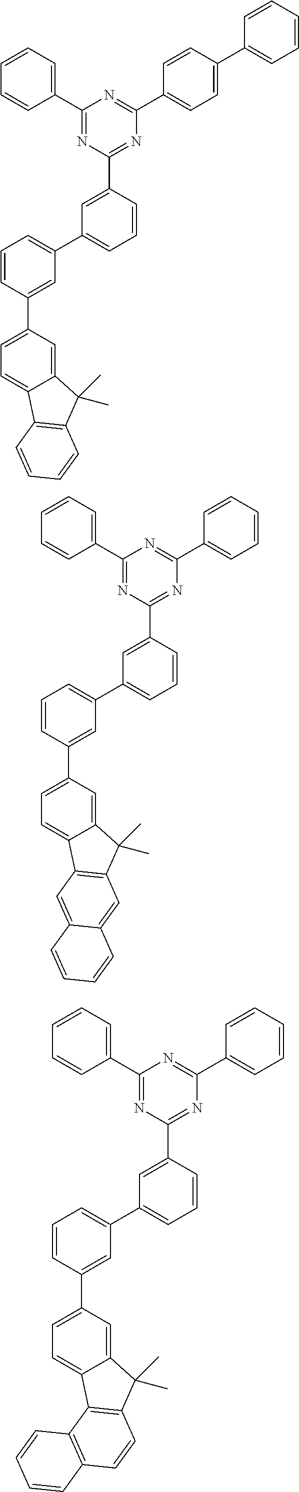

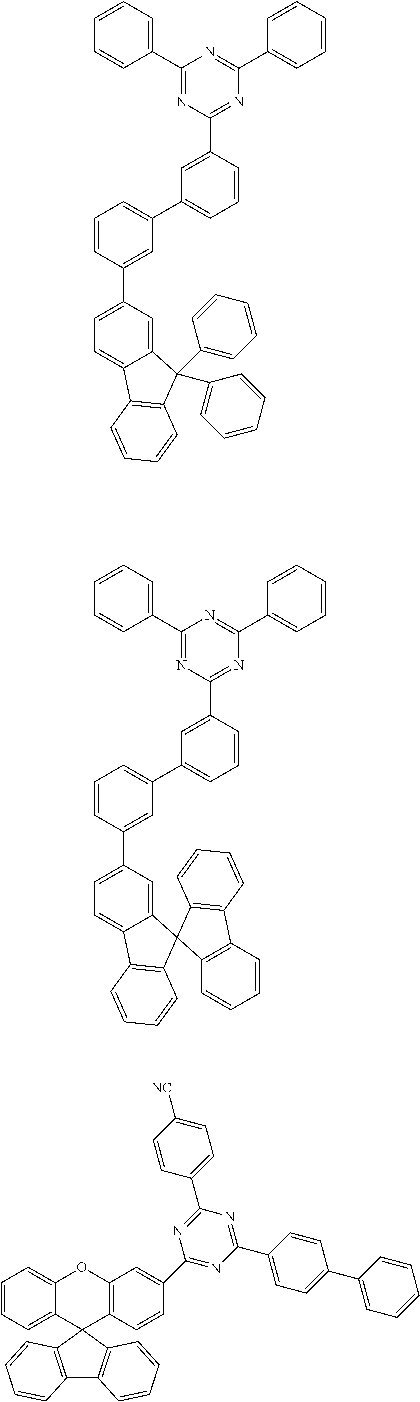 [SMILES: c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1 ; N#Cc1ccc(-c2nc(-c3ccc(-c4ccccc4)cc3)nc(-c3ccc4c(c3)Oc3ccccc3C43c4ccccc4-c4ccccc43)n2)cc1]

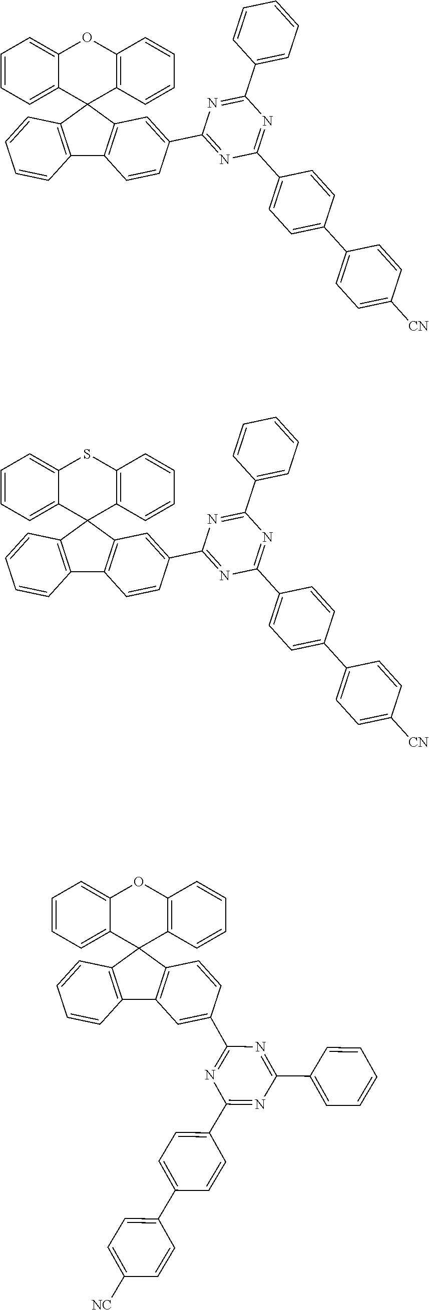 [SMILES: N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1 ; N#Cc1ccc(-c2ccc(-c3nc(-c4ccccc4)nc(-c4ccc5c(c4)-c4ccccc4C54c5ccccc5Oc5ccccc54)n3)cc2)cc1]

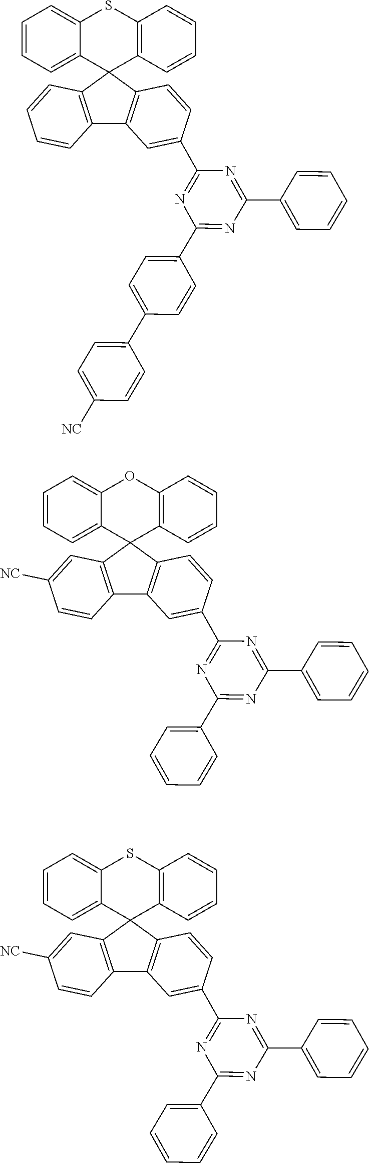 [SMILES: N#Cc1ccc(-c2ccc(-c3nc(-c4ccccc4)nc(-c4ccc5c(c4)-c4ccccc4C54c5ccccc5Sc5ccccc54)n3)cc2)cc1 ; N#Cc1ccc2c(c1)C1(c3ccccc3Oc3ccccc31)c1ccc(-c3nc(-c4ccccc4)nc(-c4ccccc4)n3)cc1-2 ; N#Cc1ccc2c(c1)C1(c3ccccc3Sc3ccccc31)c1ccc(-c3nc(-c4ccccc4)nc(-c4ccccc4)n3)cc1-2]

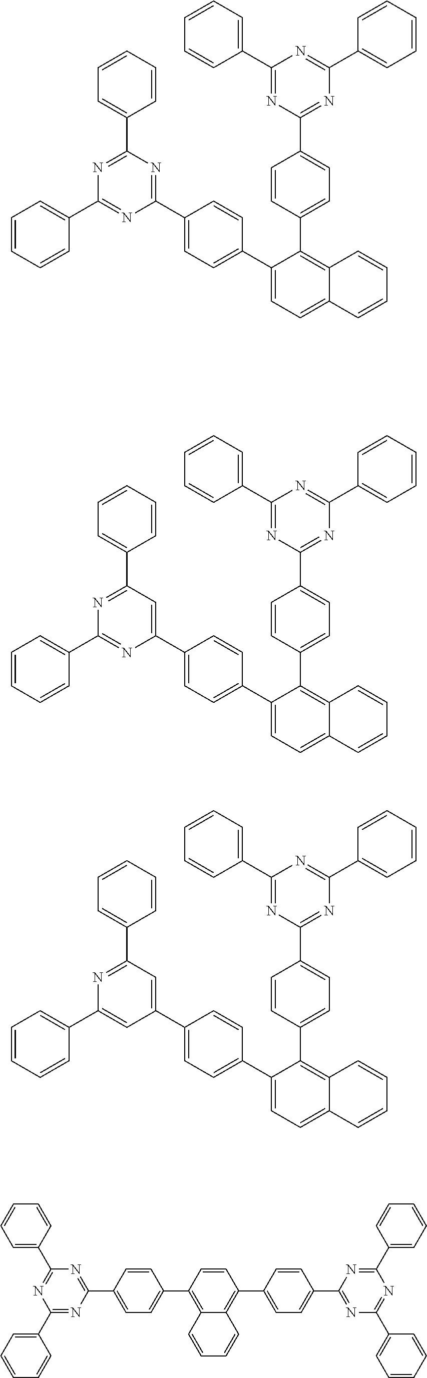 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)nc(-c3ccccc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)cc(-c3ccccc3)n2)cc1 ; c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1]

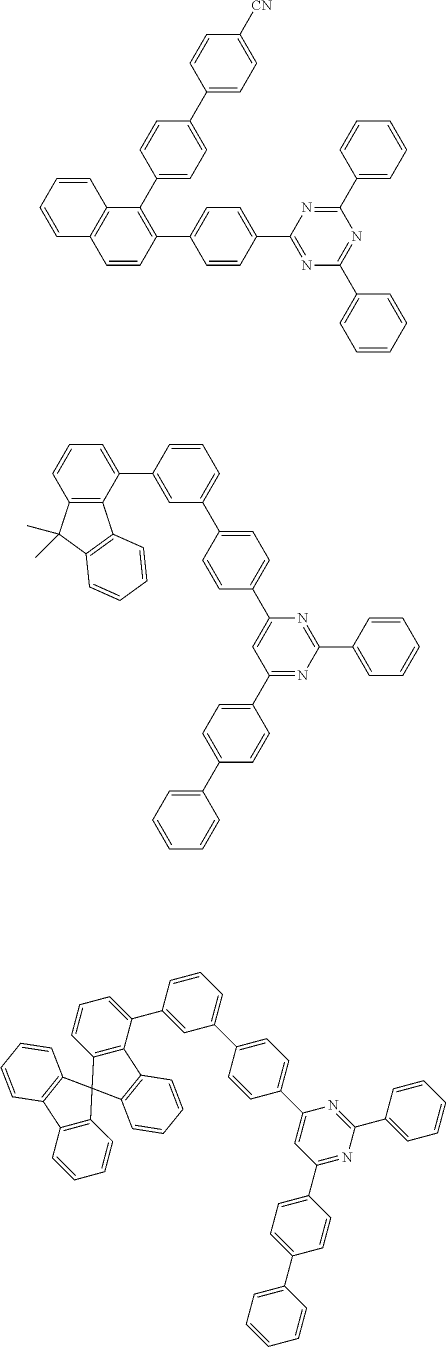 [SMILES: N#Cc1ccc(-c2ccc(-c3c(-c4ccc(-c5nc(-c6ccccc6)nc(-c6ccccc6)n5)cc4)ccc4ccccc34)cc2)cc1 ; CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1]

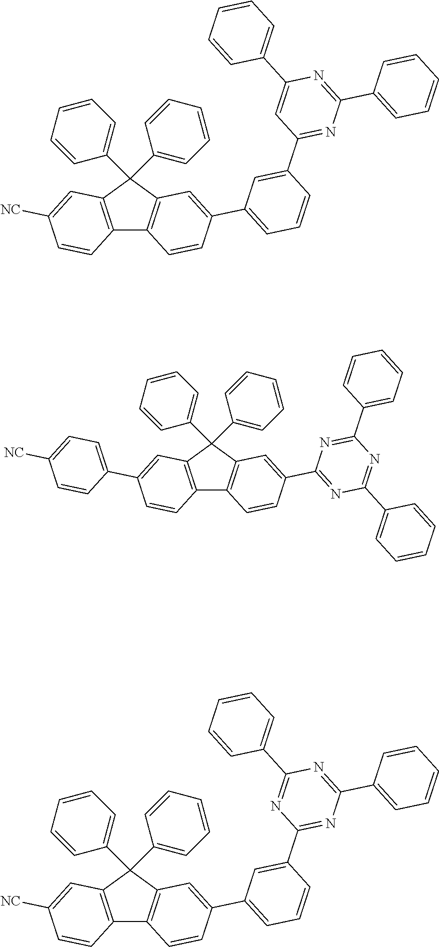 [SMILES: N#Cc1ccc2c(c1)C(c1ccccc1)(c1ccccc1)c1cc(-c3cccc(-c4cc(-c5ccccc5)nc(-c5ccccc5)n4)c3)ccc1-2 ; N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

An embodiment of the present invention may provide an organic light emitting diode including the light emitting layer as the organic layer.

Another embodiment of the present invention may provide an organic light emitting diode including a light emitting layer and a hole transport region as the organic layer.

Another embodiment of the present invention may provide an organic light emitting diode including a light emitting layer and an electron transport region as the organic layer.

Another embodiment of the present invention may provide an organic light emitting diode including a hole transport region 140 and an electron transport region 150 in addition to the light emitting layer 130 as the organic layer 105, as shown in FIG. 1.

On the other hand, an organic light emitting diode may further include an electron injection layer (not shown), a hole injection layer (not shown), etc. in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

### Detailed Description of the Embodiments

Hereinafter, the embodiments are illustrated in more detail with reference to examples. However, these examples are exemplary, and the scope of claims is not limited thereto.

Hereinafter, starting materials and reactants used in Examples and Synthesis Examples were purchased from Sigma-Aldrich Co. Ltd., TCI Inc., Tokyo chemical industry, or P&H tech as far as there in no particular comment or were synthesized by known methods.

### (Preparation of Compound for Organic Optoelectronic Device)

The compounds presented as a more specific example of the compound of the present invention were synthesized through the following steps.

### Synthesis Example 1: Synthesis of Compound A-1

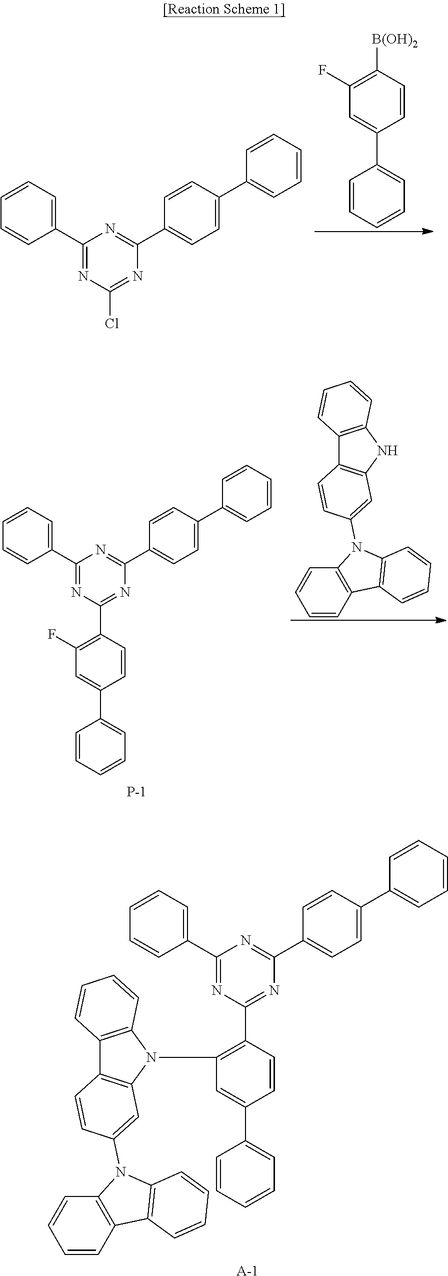

### 1st Step: Synthesis of Intermediate P-1

2-([1,1′-biphenyl]-4-yl)-4-chloro-6-phenyl-1,3,5-triazine (30 g/1.0 eq.), (3-fluoro-[1,1′-biphenyl]-4-yl)boronic acid (1.1 eq.), Pd(PPh₃)₄(0.05 eq.), and K₂CO₃ (3.0 eq.) with THF (500 mL) and distilled water (165 mL) were added to a flask and refluxed at 80° C. After 12 hours, the reaction was completed, the resultant was diluted with DCM, washed three times with brine, and dried with MgSO₄. Subsequently, 33 g of Intermediate P-1 was obtained through column chromatography.

### 2nd Step: Synthesis of Compound A-1

Intermediate P-1 (33 g/1.0 eq.), 9H-2,9′-bicarbazole (1.5 eq.), and K₃PO₄ (3.0 eq.) with DMF (500 mL) were added to a flask and refluxed at 150° C. After 12 hours, the reaction was completed, the resultant was diluted with DCM, washed three times with brine, and dried with MgSO₄. Subsequently, 30 g of Compound A-1 was obtained through column chromatography.

### Synthesis Example 2: Synthesis of Compound A-2

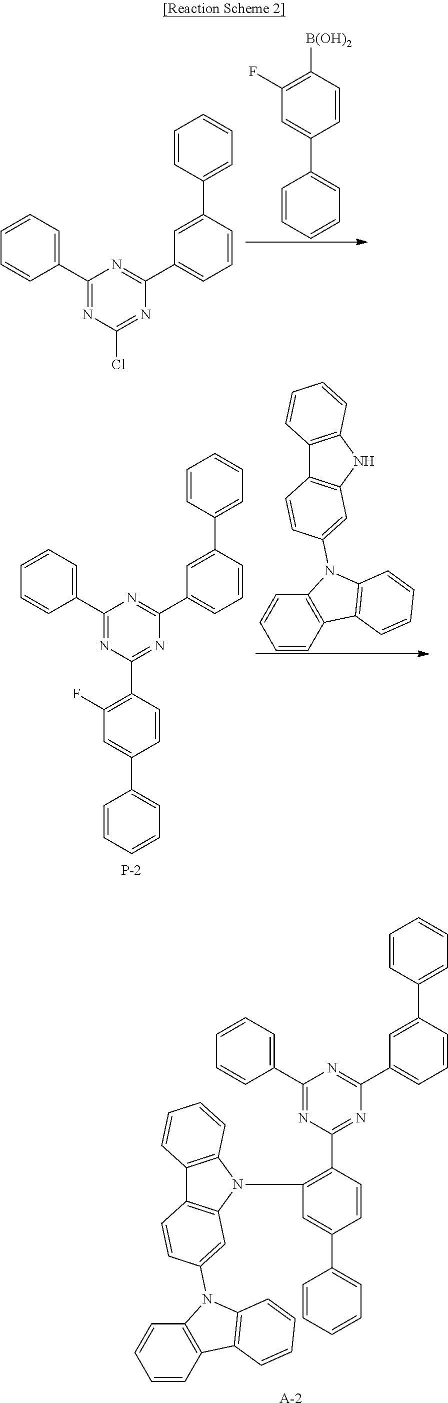

### 1st Step: Synthesis of Intermediate P-2

2-([1,1′-biphenyl]-3-yl)-4-chloro-6-phenyl-1,3,5-triazine (30 g/1.0 eq.), (3-fluoro-[1,1′-biphenyl]-4-yl)boronic acid (1.1 eq.), Pd(PPh₃)₄(0.05 eq.), and K₂CO₃ (3.0 eq.) with THF (500 mL) and distilled water (165 mL) were added to a flask and refluxed at 80° C. After 12 hours, the reaction was completed, the resultant was diluted with DCM, washed three times with brine, and dried with MgSO₄. Subsequently, 32 g of Intermediate P-2 was obtained through column chromatography.

### 2nd Step: Synthesis of Compound A-2

Intermediate P-2 (32 g/1.0 eq.), 9H-2,9′-bicarbazole (1.5 eq.), and K₃PO₄ (3.0 eq.) with DMF (500 mL) were added to a flask and refluxed at 150° C. After 12 hours, the reaction was completed, the resultant was diluted with DCM, washed three times with brine, and dried with MgSO₄. Subsequently, 26 g of Compound A-2 was obtained through column chromatography.

### Comparative Synthesis Example 1: Synthesis of Compound R-1

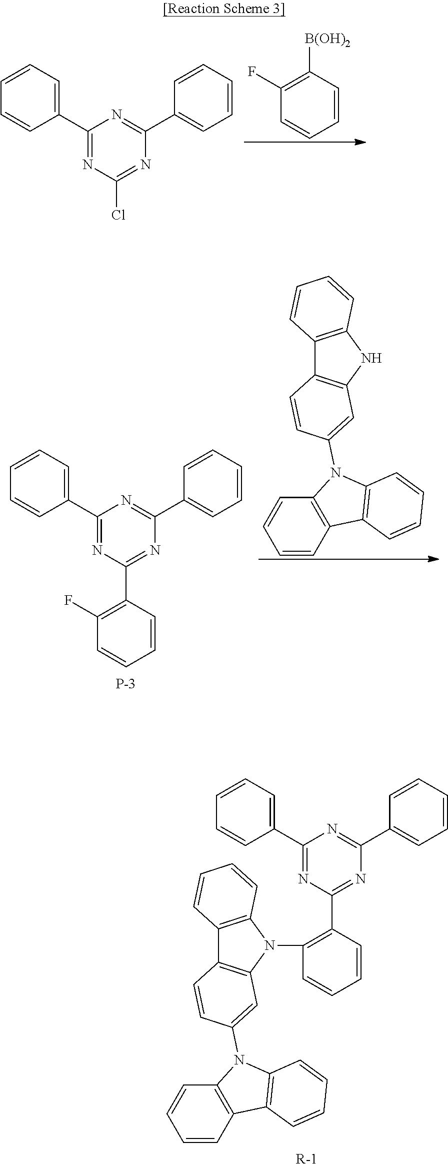

### 1st Step: Synthesis of Intermediate P-3

2-chloro-4,6-diphenyl-1,3,5-triazine (30 g/1.0 eq.), (2-fluorophenyl)boronic acid (1.1 eq.), Pd(PPh₃)₄(0.05 eq.), and K₂CO₃ (3.0 eq.) with THF (500 mL) and distilled water (165 mL) were added to a flask and refluxed at 80° C. After 12 hours, the reaction was completed, the resultant was diluted with DCM, washed three times with brine, and dried with MgSO₄. Subsequently, 25 g of Intermediate P-3 was obtained through column chromatography.

### 2nd Step: Synthesis of Compound R-1

Intermediate P-3 (25 g/1.0 eq.), 9H-2,9′-bicarbazole (1.5 eq.), and K₃PO₄ (3.0 eq.) with DMF (500 mL) were added to a flask and refluxed at 150° C. After 12 hours, the reaction was completed, the resultant was diluted with DCM, washed three times with brine, and dried with MgSO₄. Subsequently, 25 g of Compound R-1 was obtained through column chromatography.

### Comparative Synthesis Example 2: Synthesis of Compound R-2

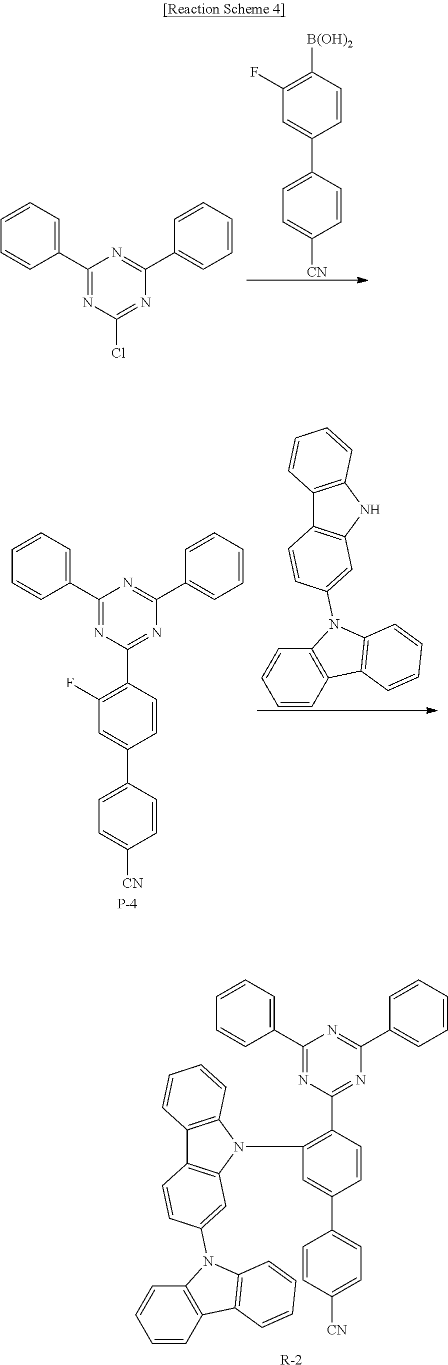

### 1st Step: Synthesis of Intermediate P-4

2-chloro-4,6-diphenyl-1,3,5-triazine (30 g/1.0 eq.), (4′-cyano-3-fluoro-[1,1′-biphenyl]-4-yl)boronic acid (1.1 eq.), Pd(PPh₃)₄(0.05 eq.), and K₂CO₃ (3.0 eq.) with THF (500 mL) and distilled water (165 mL) were added to a flask and refluxed at 80° C. After 12 hours, the reaction was completed, the resultant was diluted with DCM, washed three times with brine, and dried with MgSO₄. Subsequently, 35 g of Intermediate P-4 was obtained through column chromatography.

### 2nd Step: Synthesis of Compound R-2

Intermediate P-4 (35 g/1.0 eq.), 9H-2,9′-bicarbazole (1.5 eq.), and K₃PO₄ (3.0 eq.) with DMF (500 mL) were added to a flask and refluxed at 150° C. After 12 hours, the reaction was completed, the resultant was diluted with DCM, washed three times with brine, and dried with MgSO₄. Subsequently, 40 g of Compound R-2 was obtained through column chromatography.

### Manufacturing of Organic Light Emitting Diode

### Example 1

A glass substrate coated with a thin film of ITO (indium tin oxide) was ultrasonically cleaned with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 1350 Å to form a hole transport layer. On the hole transport layer, Compound B was deposited to a thickness of 350 Å to form a hole transport auxiliary layer, and on the hole transport auxiliary layer, Compound A-1 obtained in Synthesis Example 1 was used as a host, and 7 wt % of PhGD was used as a dopant to form a 400 Å-thick light emitting layer by vacuum deposition. Subsequently, Compound C was deposited on the light emitting layer to a thickness of 50 Å to form an electron transport auxiliary layer, and Compound D and LiQ were simultaneously vacuum-deposited in a weight ratio of 1:1 to form a 300 Å-thick electron transport layer. An organic light emitting diode was manufactured by sequentially vacuum-depositing 15 Å of LiQ and 1,200 Å of Al on the electron transport layer to form a cathode.

ITO/Compound A (3% NDP-9 doping, 100 Å)/Compound A (1350 Å)/Compound B (350 Å)/EML [93 wt % of host (Compound A-1): 7 wt % of PhGD] (400 Å)/Compound C (50 Å)/Compound D: LiQ (300 Å)/LiQ (15 Å)/Al (1200 Å).

Compound A: N-(biphenyl-4-yl)-9,9-dimethyl-N-(4-(9-phenyl-9H-carbazol-3-yl)phenyl)-9H-fluoren-2-amine

Compound B: N-[4-(4-Dibenzofuranyl)phenyl]-N-[4-(9-phenyl-9H-fluoren-9-yl)phenyl]1[1,1′-biphenyl]-4-amine

Compound C: 2,4-Diphenyl-6-(4′,5′,6′-triphenyl[1,1′:2′,1″:3″,1′″:3′″,1′″-quinquephenyl]-3′″-yl)-1,3,5-triazine

Compound D: 2-(1,1′-Biphenyl-4-yl)-4-(9,9-diphenylfluoren-4-yl)-6-phenyl-1,3,5-triazine

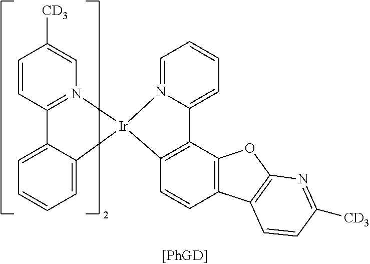

### Example 2 and Comparative Examples 1 and 2

Each diode of Example 2 and Comparative Examples 1 and 2 was manufactured in the same manner as Example 1, except that the host was changed as shown in Table 1.

### Evaluation

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change Luminance was measured by using a luminance meter (Minolta Cs-1000A), while the voltage of the organic light emitting diodes was increased from 0 V to 10 V.

### (3) Measurement of Luminous Efficiency

Luminous efficiency (cd/A) at the same current density (10 mA/cm²) were calculated by using the luminance and current density from (1) and (2) above.

Relative values based on the luminous efficiency of Comparative Example 1 are shown in Table 1.

### (4) Measurement of Life-span

T95 life-spans of the organic light emitting diodes were measured as a time when their luminance decreased down to 95% relative to the initial luminance (cd/m²) after emitting light with the initial luminance of 2,4000 cd/m² and measuring their luminance decrease depending on time with a Polanonix life-span measurement system.

Relative values based on the T95 life-span of Comparative Example 1 are shown in Table 1.

### (5) Measurement of Driving Voltage

The driving voltage of each diode were measured at 15 mA/cm² using a current-voltmeter (Keithley 2400), and the results were obtained.

Relative values based on the driving voltage of Comparative Example 1 are shown in Table 1.

TABLE 1

| | | Driving | Luminous | Life-span |
|---|---|---|---|---|
| | | voltage | efficiency | T95 |
| | Host | (%) | (%) | (%) |
| Example 1 | A-1 | 94.6 | 102.1 | 129.5 |
| Example2 | A-2 | 96.9 | 101.9 | 121.0 |
| Comparative Example1 | R-1 | 100.0 | 100.0 | 100.0 |
| Comparative Example2 | R-2 | 118.5 | 86.9 | 72.3 |

Referring to Table 1, the driving voltages, luminous efficiency, and life-span characteristics of the organic light emitting diodes to which the compounds according to Example of the present invention are applied are significantly improved compared to the organic light emitting diodes according to Comparative Examples.

While this invention has been described in connection with what is presently considered to be practical example embodiments, it is to be understood that the invention is not limited to the disclosed embodiments. On the contrary, it is intended to cover various modifications and equivalent arrangements included within the spirit and scope of the appended claims.

1. A compound for an organic optoelectronic device, the compound being represented by Chemical Formula 1: 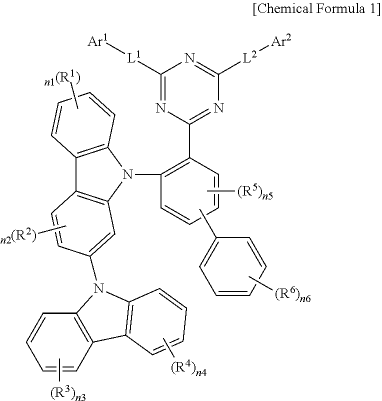
wherein, in Chemical Formula 1,
Ar¹ and Ar² are each independently a substituted or unsubstituted C6 to C30 aryl group,
L¹ and L² are each independently a single bond or a substituted or unsubstituted C6 to C30 arylene group,
R¹ to R⁴ are each independently hydrogen, deuterium, or a substituted or unsubstituted C6 to C30 aryl group,
R⁵ and R⁶ are each independently hydrogen or deuterium,
n¹, n³, and n⁴ are each independently an integer of 1 to 4,
n2 and n5 are each independently integer of 1 to 3, and
n6 is an integer of 1 to 5.

2. The compound for an organic optoelectronic device as claimed in claim 1, wherein:
the compound is represented by Chemical Formula 1-1: 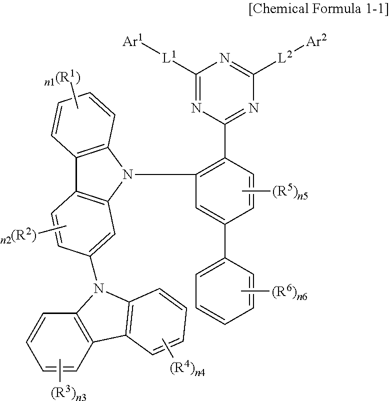
in Chemical Formula 1-1, Ar¹, Ar², R¹ to R⁶, L¹, L², and n1 to n6 are defined the same as those of Chemical Formula 1.

3. The compound for an organic optoelectronic device as claimed in claim 1, wherein R¹ to R⁴ are each independently hydrogen, deuterium, a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, or a substituted or unsubstituted triphenylene group.

4. The compound for an organic optoelectronic device as claimed in claim 1, wherein Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted dibenzofuranyl group, or a substituted or unsubstituted dibenzothiophenyl group.

5. The compound for an organic optoelectronic device as claimed in claim 1, wherein:
moieties *-L¹-Ar¹ and *-L²-Ar² are each independently a moiety of Group I: 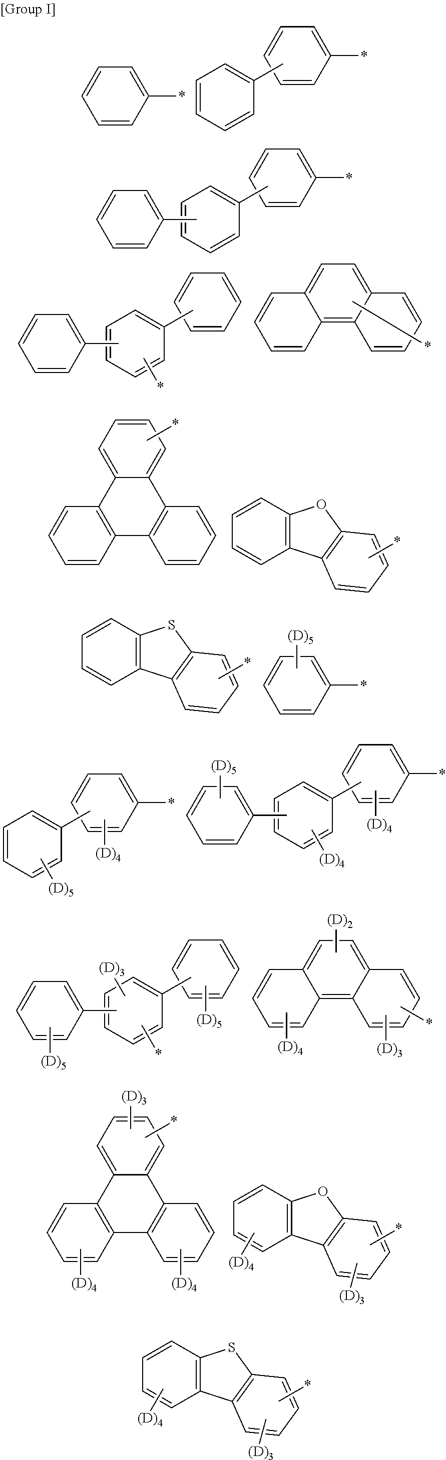
in Group I, * is a linking point.

6. The compound for an organic optoelectronic device as claimed in claim 1, wherein the compound is a compound of Group 1:

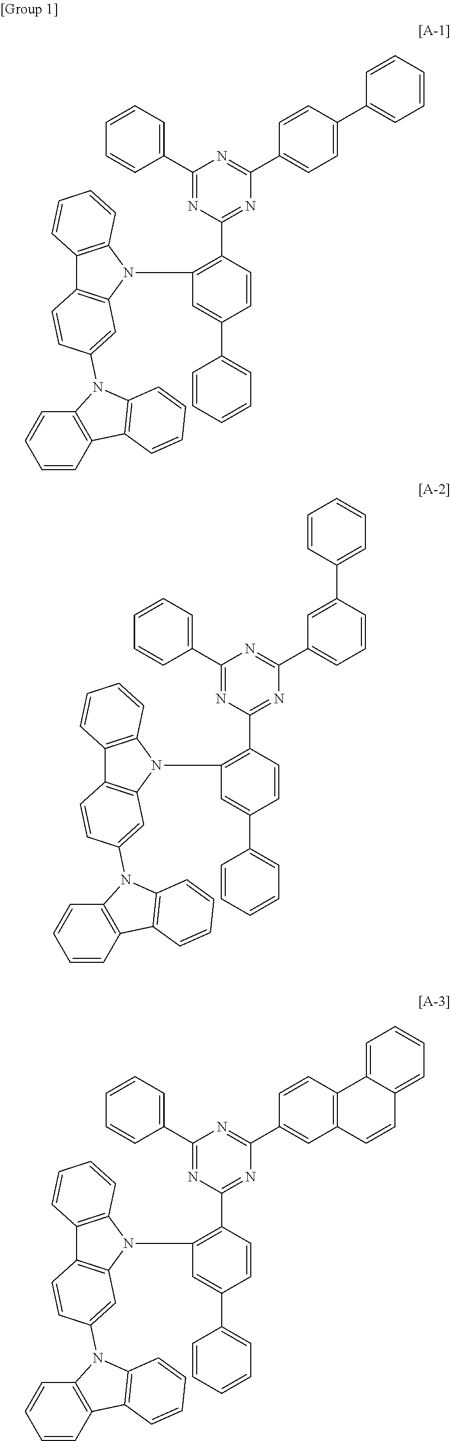 [SMILES: c1ccc(-c2ccc(-c3nc(-c4ccccc4)nc(-c4ccc(-c5ccccc5)cc4-n4c5ccccc5c5ccc(-n6c7ccccc7c7ccccc76)cc54)n3)cc2)cc1 ; c1ccc(-c2cccc(-c3nc(-c4ccccc4)nc(-c4ccc(-c5ccccc5)cc4-n4c5ccccc5c5ccc(-n6c7ccccc7c7ccccc76)cc54)n3)c2)cc1 ; c1ccc(-c2ccc(-c3nc(-c4ccccc4)nc(-c4ccc5c(ccc6ccccc65)c4)n3)c(-n3c4ccccc4c4ccc(-n5c6ccccc6c6ccccc65)cc43)c2)cc1]

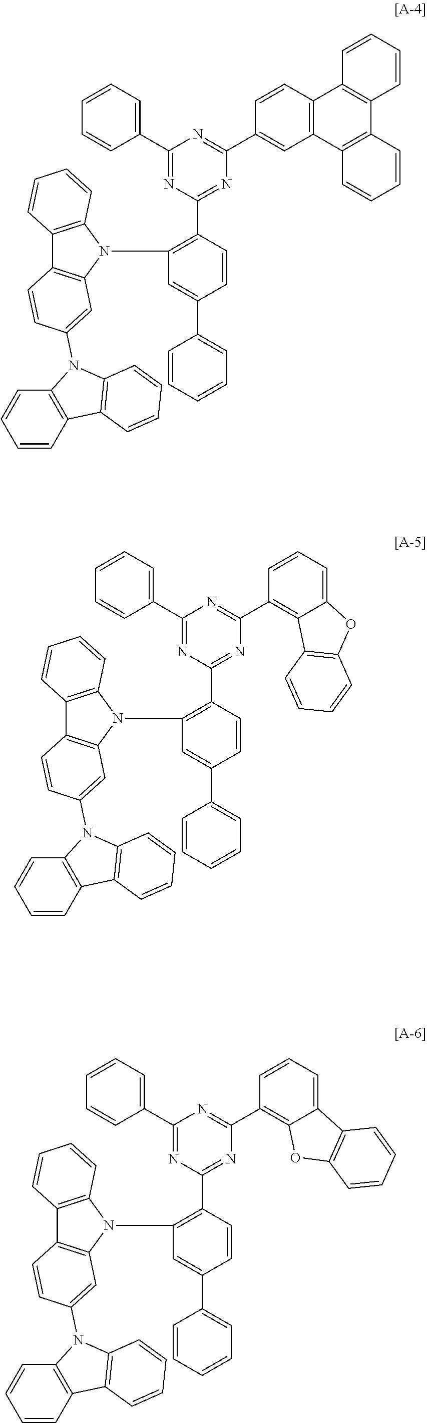 [SMILES: c1ccc(-c2ccc(-c3nc(-c4ccccc4)nc(-c4ccc5c6ccccc6c6ccccc6c5c4)n3)c(-n3c4ccccc4c4ccc(-n5c6ccccc6c6ccccc65)cc43)c2)cc1 ; c1ccc(-c2ccc(-c3nc(-c4ccccc4)nc(-c4cccc5oc6ccccc6c45)n3)c(-n3c4ccccc4c4ccc(-n5c6ccccc6c6ccccc65)cc43)c2)cc1 ; c1ccc(-c2ccc(-c3nc(-c4ccccc4)nc(-c4cccc5c4oc4ccccc45)n3)c(-n3c4ccccc4c4ccc(-n5c6ccccc6c6ccccc65)cc43)c2)cc1]

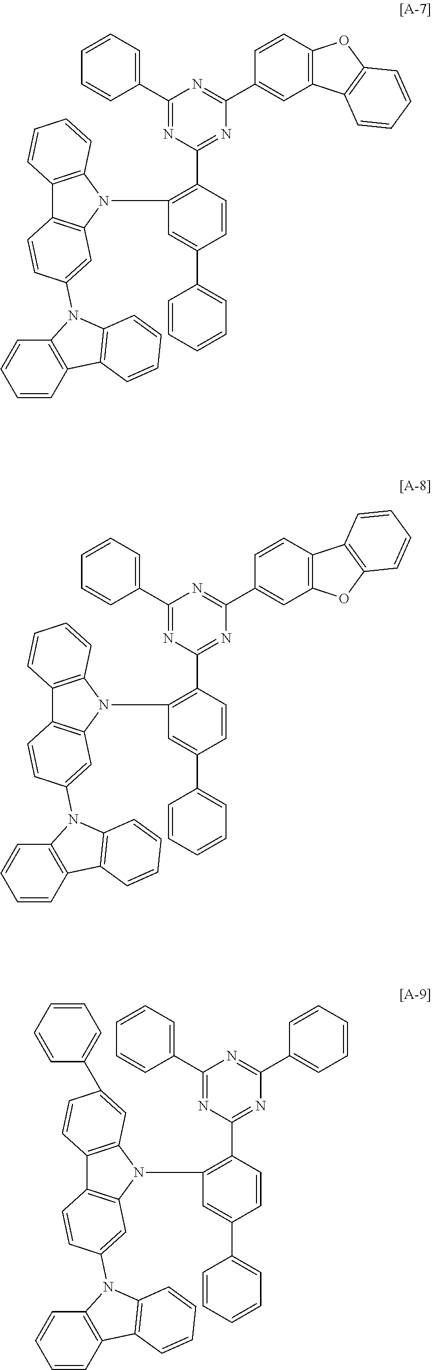 [SMILES: c1ccc(-c2ccc(-c3nc(-c4ccccc4)nc(-c4ccc5oc6ccccc6c5c4)n3)c(-n3c4ccccc4c4ccc(-n5c6ccccc6c6ccccc65)cc43)c2)cc1 ; c1ccc(-c2ccc(-c3nc(-c4ccccc4)nc(-c4ccc5c(c4)oc4ccccc45)n3)c(-n3c4ccccc4c4ccc(-n5c6ccccc6c6ccccc65)cc43)c2)cc1 ; c1ccc(-c2ccc(-c3nc(-c4ccccc4)nc(-c4ccccc4)n3)c(-n3c4cc(-c5ccccc5)ccc4c4ccc(-n5c6ccccc6c6ccccc65)cc43)c2)cc1]

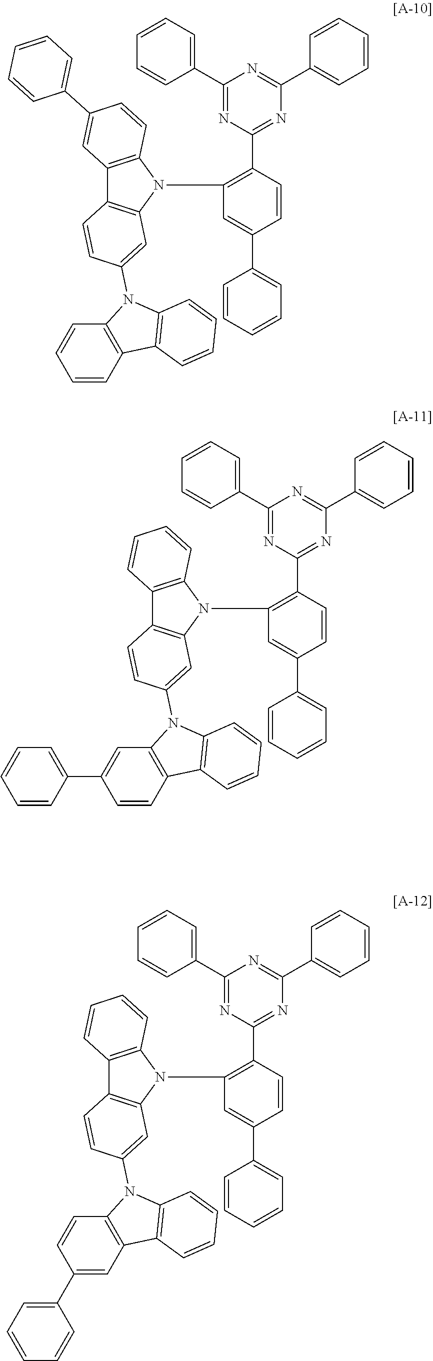 [SMILES: c1ccc(-c2ccc(-c3nc(-c4ccccc4)nc(-c4ccccc4)n3)c(-n3c4ccc(-c5ccccc5)cc4c4ccc(-n5c6ccccc6c6ccccc65)cc43)c2)cc1 ; c1ccc(-c2ccc(-c3nc(-c4ccccc4)nc(-c4ccccc4)n3)c(-n3c4ccccc4c4ccc(-n5c6ccccc6c6ccc(-c7ccccc7)cc65)cc43)c2)cc1 ; c1ccc(-c2ccc(-c3nc(-c4ccccc4)nc(-c4ccccc4)n3)c(-n3c4ccccc4c4ccc(-n5c6ccccc6c6cc(-c7ccccc7)ccc65)cc43)c2)cc1]

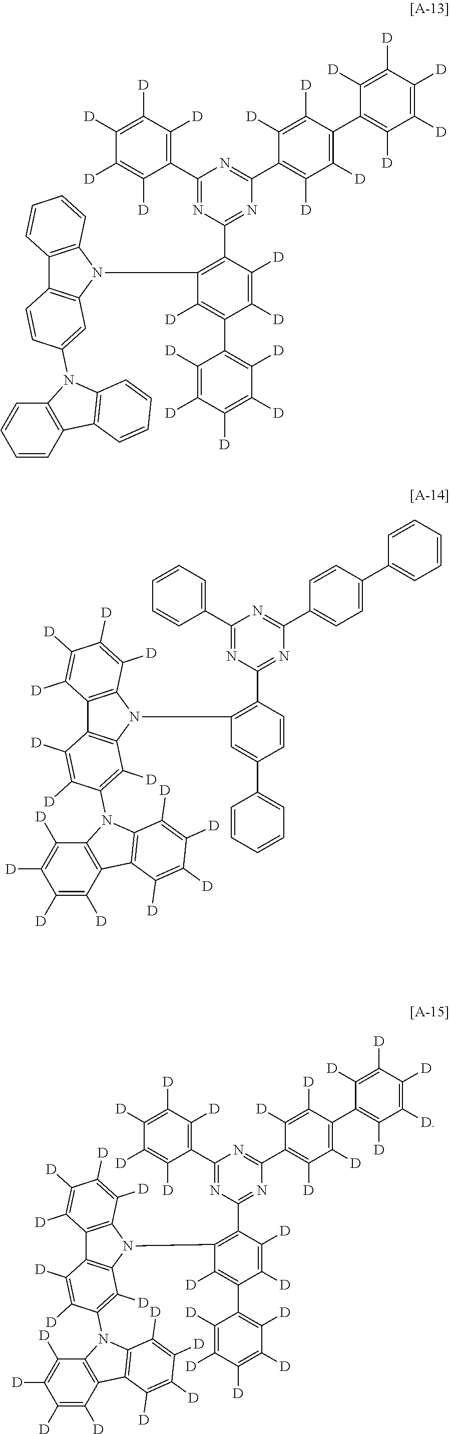

7. An organic photoelectronic device, comprising:
an anode and a cathode facing each other, and
at least one organic layer between the anode and the cathode,
wherein the at least one organic layer includes the compound for an organic optoelectronic device as claimed in claim 1.

8. The organic photoelectronic device as claimed in claim 7, wherein:
the at least one organic layer includes a light emitting layer, and
the light emitting layer includes the compound.

9. A display device comprising the organic photoelectronic device as claimed in claim 7.
